(19) United States
(12) Patent Application Publication
(10) Pub. No.: US 2003/0211971 A1
(43) Pub. Date: Nov. 13, 2003

(54) Compositions and methods for prevention and treatment of primary and metastatic neoplastic diseases and infectious diseases with compositions comprising unfractionated cellular proteins

(76) Inventor: Pramod K. Srivastava, Avon, CT (US)

Correspondence Address: PENNIE AND EDMONDS, 1155 AVENUE OF THE AMERICAS, NEW YORK, NY, 100362711

(21) Appl. No.: 10/380,442

(22) PCT Filed: Sep. 17, 2001
(86) PCT No.: PCT/US01/28841

(51) Int. Cl. A61K 39/00; A61K 38/00

(52) U.S. Cl. 514/2; 424/277.1

(57) ABSTRACT

The present invention relates to methods and compositions for the prevention and treatment of infectious diseases, and primary and metastatic neoplastic diseases, including, but not limited to human sarcomas and carcinomas. In the practice of the prevention and treatment of infectious diseases and cancer, compositions comprising unfractionated cellular proteins are used to augment the immune response to genotoxic and nongenotoxic factors, tumors and infectious agents.

This application claims benefit of U.S. Provisional Application Serial No. 60/233,174, filed Sep. 15, 2000, which is incorporated by reference herein in its entirety.

This invention was made with government support under grant numbers CA44786 and CA64394 awarded by the National Institutes of Health. The government has certain rights in the invention.

### 1. INTRODUCTION

The present invention relates to methods and compositions for the prevention and treatment of infectious diseases, and primary and metastatic neoplastic diseases, including, but not limited to human sarcomas and carcinomas. In the practice of the prevention and treatment of infectious diseases and cancer, compositions comprising unfractionated cellular proteins are used to augment the immune response to genotoxic and nongenotoxic factors, tumors and infectious agents.

### 2. BACKGROUND OF THE INVENTION

The era of tumor immunology began with experiments by Prehn and Main, who showed that antigens on the methylcholanthrene (MCA)-induced sarcomas were tumor specific in that transplantation assays could not detect these antigens in normal tissue of the mice (Prehn, R. T., et al., 1957, J. Natl. Cancer Inst. 18:769-778). This notion was confirmed by further experiments demonstrating that tumor specific resistance against MCA-induced tumors can be elicited in the autochthonous host, that is, the mouse in which the tumor originated (Klein, G., et al., 1960, Cancer Res. 20:1561-1572).

In subsequent studies, tumor specific antigens were also found on tumors induced with other chemical or physical carcinogens or on spontaneous tumors (Kripke, M. L., 1974, J. Natl. Cancer Inst. 53:1333-1336; Vaage, J., 1968, Cancer Res. 28:2477-2483; Carswell, E. A., et al., 1970, J. Natl. Cancer Inst. 44:1281-1288). Since these studies used protective immunity against the growth of transplanted tumors as the criterion for tumor specific antigens, these antigens are also commonly referred to as “tumor specific transplantation antigens” or “tumor specific rejection antigens.” Several factors can greatly influence the immunogenicity of the tumor induced, including, for example, the specific type of carcinogen involved, immunocompetence of the host and latency period (Old, L. J., et al., 1962, Ann. N.Y. Acad. Sci. 101:80-106; Bartlett, G. L., 1972, J. Natl. Cancer Inst. 49:493-504).

Most, if not all, carcinogens are mutagens which may cause mutation, leading to the expression of tumor specific antigens (Ames, B. N., 1979, Science 204:587-593; Weisburger, J. H., et al., 1981, Science 214:401-407). Some carcinogens are immunosuppressive (Malmgren, R. A., et al., 1952, Proc. Soc. Exp. Biol. Med. 79:484-488). Experimental evidence suggests that there is a constant inverse correlation between immunogenicity of a tumor and latency period (time between exposure to carcinogen and tumor appearance) (Old, L. J., et al., 1962, Ann. N.Y. Acad. Sci. 101:80-106; and Bartlett, G. L., 1972, J. Natl. Cancer Inst. 49:493-504). Other studies have revealed the existence of tumor specific antigens that do not lead to rejection, but, nevertheless, can potentially stimulate specific immune responses (Roitt, I., Brostoff, J and Male, D., 1993, Immunology, 3rd ed., Mosby, St. Louis, pps. 17.1-17.12).

Even before tumor-specific antigens had been identified, attempts had been made, as early as 1777, to develop cancer vaccines derived from neoplastic tissue samples (Oettgen, H. F., and Old, L. J., 1991, The History of Cancer Immunotherapy, in Introduction to the Biologic Therapy of Cancer, DeVitta, V. T., Hellman, S., and Rosenberg, S. A. Editors, Lippincott, Philadelphia, pp. 87-119). Individuals have inoculated themselves and others with compositions comprising cancer tissue, extracts from cancer tissue, cultured cancer cells, and tumor cells modified by viral infection, enzymatic digestion, or chemical treatment (Oettgen, H. F., and Old, L. J., 1991, The History of Cancer Immunotherapy, in Introduction to the Biologic Therapy of Cancer, DeVitta, V. T., Hellman, S., and Rosenberg, S. A. Editors, Lippincott, Philadelphia, pp. 87-119). Results provided by these experiments have generally been deemed inconclusive, at best (Oettgen, H. F., and Old, L. J., 1991, The History of Cancer Immunotherapy, in Introduction to the Biologic Therapy of Cancer, DeVitta, V. T., Hellman, S., and Rosenberg, S. A. Editors, Lippincott, Philadelphia, pp. 87-119). However, there have been anecdotal reports suggesting that other approaches may be therapeutically effective (Cassel, W. A., et al., 1983, Cancer,52: 856-60; Humphrey, L. J., et al., 1984, Journal of Surgical Oncology, 25:303-05).

### 2.1. Immunogenicity of Tumor Cells, Tumor Cell Lysates and Sub-Cellular Fractions of Tumor Cell Lysates

Sparks et al. developed an adjuvant chemoimmunotherapy program for the treatment of breast cancer comprising the administration of cyclophosphamide, methotrexate, fluorouracil and BCG vaccine either with or without a second vaccine consisting of irradiated, allogeneic breast cancer cells (Sparks, F. C., et al., 1976, Arch Surg, 111, 1057-62). The tumor cell vaccine was made up of 3.5×10⁷ irradiated cells from each of three different breast carcinoma cell lines: MDA-MB-231, MDA-MB-157, and NBL-374B. The authors indicated that the preliminary data obtained from this approach suggested that adjuvant chemoimmunotherapy may affect the natural progression of breast carcinoma (Sparks, F. C., et al., 1976, Arch Surg, 111, 1057-62).

Hughes et al. reported the use of homogenized, fractionated tumor tissue as a vaccine for clinical cancer immunotherapy (Hughes, L. E. et al., 1970, Cancer, 26(2):269-78). Subcellular extracts were prepared by homogenizing autologous tumor samples and then breaking the isolated cells by sonication. Lysed material was subjected to low speed centrifugation (600×g, 10 min.), to provide a first supernatant that was recentrifuged at 8500×g for 10 minutes to provide the mitochondrial pellet and the second supernatant. The latter supernatant fraction was centrifuged (40,000×g, 45 min.), yielding a microsomal pellet and the third supernatant, which was referred to as the “cell sap.” Fifteen patients were treated with a tumor extract composition comprising cell sap combined with the microsomal pellet while five patients were treated with a composition comprising a combination of the microsomal and mitochondrial fractions. The clinical results obtained in these studies were deemed to fall within the limits of the natural course. of this disease, indicating that no apparent benefit had been provided by this treatment (Hughes, L. E. et al., 1970, Cancer, 26(2) :269-78).

Humphrey et al. administered a tumor extract, referred to as a “tumor associated antigen preparation,” for the treatment of post-surgical, melanoma patients (Humphrey, L. J., et al., 1984, Journal of Surgical Oncology, 25:303-05). Tumor tissue was homogenized as a 20% w/v suspension in a 0.25M sucrose buffer and centrifuged (102,000×g, 74 min.). The supernatant obtained was concentrated two-fold against a UM-10 Amicon filter and then frozen. One milliliter aliquots were administered weekly for eight weeks and then quarterly for two years. Since there were no control patients involved in this study, patient survival was compared to historical data. As measured by this standard, the authors suggested that the immunotherapy had modified the host's response to the melanoma and warranted further exploration (Humphrey, L. J., et al., 1984, Journal of Surgical Oncology, 25:303-05).

Cassel et al. have described a cell extract prepared by infecting cultured malignant melanoma tumor cells with Newcastle disease virus and collecting the resulting lysate. The “viral oncolysate” was clarified by centrifugation at 700×g for 10 minutes and concentrated ten-fold against a PM-10 membrane in a Diaflo Cell (Amicon Corp.), providing an extract in which one milliliter of this ten-fold concentrate comprises approximately 8×10⁶ cell equivalents (Cassel, W. A. et al., 1977, Cancer 40: 672-79). Two and one half milliliters (˜2×10⁷ cell equivalents) of viral oncolysate were administered to patients with Stage II malignant melanoma. Each dose of lysate represented a mixture of three components: (1) 0.5 ml of lysate prepared from cell line MRD; (2) one ml of lysate prepared from either cell line BMCL or M40; and (3) 1 ml of one lysate prepared from a cell line selected from the group consisting of seven specified cell lines (the choice was rotated with each patient visit). Where possible, autologous viral oncolysates were prepared from tissue melanoma tissue taken from the patient, and in those instances, the autologous material was used in place of the third component of the mixture administered. The frequency with which patients treated in. this manner remained free of disseminated disease was substantially improved as compared to the control patient population (Cassel, W. A., et al., 1983, Cancer 52: 856-60).

Rogers et al. have described fractionation of tumor cell extracts for the isolation of tumor-associated transplantation antigens (Rogers et al., 1981, Int. J. Cancer, 27: 789-96). Subcellular fractions were derived from cell lysates prepared from two highly immunogenic methylcholanthrene-induced murine sarcomas, Meth A and CI-4. Cells were suspended in buffer, allowed to swell at 4° C., mechanically disrupted, and then centrifuged at 100,000×g for one hour. Plasma membrane fractions were isolated from clarified cell extracts by ultracentrifugation in a discontinuous sucrose-dextran T40 gradient.

Treatment of mice with 1-2×10⁵ cell equivalents of material prepared from the plasma membrane fraction or from the 100,000×g pellet of Meth A cells provided substantial protection against the subsequent challenge of those mice with Meth A ascites cells. In a similar manner, subcellular fractions prepared from CI-4 cells protected inoculated mice against the subsequent challenge with CI-4 cells. In addition, even though the subcellular distribution of tumor-associated transplantation antigens in Meth A cells was found to vary from one experiment to another, substantial tumor-inhibiting activity was observed in the cell cytosolic fraction (Rogers et al., 1981, Int. J. Cancer, 27: 789-96).

DuBois et al. demonstrated the presence of a soluble tumor-associated tumor antigen in Meth A murine sarcoma cells in a fractionated cell lysate (DuBois, G. C., et al., 1980, Cancer Research, 40:4204-08). Meth A cells were separated from ascites fluid by low-speed centrifugation, washed, and mechanically disrupted to provide a crude lysate, which was centrifuged at 100,000×g for fifty minutes. The high-speed supernatant was fractionated using selective precipitation with ammonium sulfate (0-55% cut). Precipitated proteins were resuspended in buffer and the resulting clarified solution concentrated against a Diaflo PM-30 membrane (Amicon Corporation, Lexington, Mass.). The concentrated material was size-fractionated on a Sephacryl S-200 column (Pharmacia Fine Chemicals, Inc., Piscataway, N.J.). In vivo tumor rejection assays performed on individual fractions of the eluate of that column indicated that Meth A tumor-specific tumor associated transplantation antigen eluted from the Sephacryl S-200 column in a molecular weight range of 43,000 to 67,000.

Srivastava et al. demonstrated the presence of an additional tumor-specific tumor rejection antigen, a 96,000 molecular weight glycoprotein, that could be isolated from chemically-induced murine sarcoma cell lines, including Meth A and CMS5 (Srivastava, P. K., 1986, Proc. Natl. Acad. Sci. 83:3407-11). Srivastava et al. also demonstrated that immunization of mice with unfractionated cytosol corresponding 5×10⁷ cell equivalents enhanced tumor growth when the treated animals were subsequently challenged with Meth A cells (Srivastava, P. K., 1986, Proc. Natl. Acad. Sci. 83:3407-11). However, fractionation of the cytosol allowed the tumor-enhancing activity to be separated from the tumor-rejection activity (Srivastava, P. K., 1986, Proc. Natl. Acad. Sci. 83:3407-11).

### 2.2. Tumor-Specific Immunogenicities of Heat Shock/Stress Proteins hsp70, hsp90 and gp96

Srivastava et al. demonstrated immune response to methylcholanthrene-induced sarcomas of inbred mice (1988, Immunol. Today 9:78-83). In these studies it was found that the molecules responsible for the individually distinct immunogenicity of these tumors were identified as cell-surface glycoproteins of 96 kDa (gp96) and intracellular proteins of 84 to 86 kDa (Srivastava, P. K., et al., 1986, Proc. Natl. Acad. Sci. USA 83:3407-3411; Ullrich, S. J., et al., 1986, Proc. Natl. Acad. Sci. USA 83:3121-3125. Immunization of mice with gp96 or p84/86 isolated from a particular tumor rendered the mice immune to that particular tumor, but not to antigenically distinct tumors. Isolation and characterization of genes encoding gp96 and p84/86 revealed significant homology between them, and showed that gp96 and p84/86 were, respectively, the endoplasmic reticular and cytosolic counterparts of the same heat shock proteins (Srivastava, P. K., et al., 1988, Immunogenetics 28:205-207; Srivastava, P. K., et al., 1991, Curr. Top. Microbiol. Immunol. 167:109-123). Further, hsp70 was shown to elicit immunity to the tumor from which it was isolated but not to antigenically distinct tumors. However, hsp70 depleted of peptides was found to lose its immunogenic activity (Udono, M., and Srivastava, P. K., 1993, J. Exp. Med. 178:1391-1396). These observations suggested that the heat shock proteins are not immunogenic per se, but are carriers of antigenic peptides that elicit specific immunity to cancers (Srivastava, P. K., 1993, Adv. Cancer Res. 62:153-177).

### 2.3. Pathobiology of Cancer

Cancer is characterized primarily by an increase in the number of abnormal cells derived from a given normal tissue, invasion of adjacent tissues by these abnormal cells, and lymphatic or blood-borne spread of malignant cells to regional lymph nodes and to distant sites (metastasis). Clinical data and molecular biologic studies indicate that cancer is a multistep process that begins with minor preneoplastic changes, which may under certain conditions progress to neoplasia.

Pre-malignant abnormal cell growth is exemplified by hyperplasia, metaplasia, or most particularly, dysplasia (for review of such abnormal growth conditions, see Robbins and Angell, 1976, Basic Pathology, 2d Ed., W. B. Saunders Co., Philadelphia, pp. 68-79.) Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

The neoplastic lesion may evolve clonally and develop an increasing capacity for invasion, growth, metastasis, and heterogeneity, especially under conditions in which the neoplastic cells escape the host's immune surveillance (Roitt, I., Brostoff, J. and Kale, D., 1993, Immunology, 3rd ed., Mosby, St. Louis, pps. 17.1-17.12).

### 2.4. Immunotherapy

Four basic cell types whose function has been associated with antitumor cell immunity and the elimination of tumor cells from the body are: i) B-lymphocytes which secrete immunoglobulins into the blood plasma for identifying and labeling the nonself invader cells; ii) monocytes which secrete the complement proteins which are responsible for lysing and processing the immunoglobulin-coated target invader cells; iii) natural killer lymphocytes having two mechanisms for the destruction of tumor cells-antibody-dependent cellular cytotoxicity and natural killing; and iv) T-lymphocytes possessing antigen-specific receptors wherein each T-lymphocyte clone having the capacity to recognize a tumor cell carrying complementary marker molecules (Schreiber, H., 1989, in Fundamental Immunology (ed). W. E. Paul, pp. 923-955).

Several factors can influence the immunogenicity of tumors induced. These factors include dose of carcinogen, immunocompetence of the host, and latency period. Immunocompetence of the host during the period of cancer induction and development can allow the host to respond to immunogenic tumor cells. This may prevent the outgrowth of these cells or select far less immunogenic escape variants that have lost their respective rejection antigen. Conversely, immunosuppression or immune deficiency of the host during carcinogenesis or tumorigenesis may allow growth of highly immunogenic tumors (Schreiber, H., 1989, in Fundamental Immunology (ed). W. E. Paul, pp. 923-955).

Four major types of cancer immunotherapy are currently being explored: i) active immunotherapy, e.g., administration of whole attenuated tumor cells or purified tumor antigens; ii) adoptive cellular immunotherapy; iii) in vivo manipulation of patient plasma to remove blocking factors or add tumoricidal factors; and iv) in vivo administration of biological response modifiers (e.g., interferons (IFN; IFN-alpha and IFN-gamma), interleukins (IL; IL-2, IL-4 and IL-6), colony-stimulating factors, tumor necrosis factor (TNF), monoclonal antibodies and other immunopotentiating agents, such as Corynebacterium parvum (C. parvum) (Rosenberg, 2001, In Cancer Principles and Practice of Oncology, 6^{th} Edition, edited by DeVita et al., Chapter 18, Lippincott, Williams and Wilkins, Philadephia; Kopp, W. C., et al., 1994, Cancer Chemotherapy and Biol. Response Modifiers 15:226-286). There is little doubt that immunotherapy of cancer as it stands is falling short of the hopes invested in it. Although numerous immunotherapeutic approaches have been tested, few of these procedures have proved to be effective as the sole or even as an adjunct form of cancer prevention and treatment.

### 2.4.1. Interleukins (IL-2, IL-4 and IL-6)

IL-2 has significant antitumor activity in a small percentage of patients with renal cell carcinoma and melanoma. Investigators continue to search for IL-2 based regimens that will increase the response rates in IL-2 responsive tumors, but, for the most part, have neither defined new indications nor settled fundamental issues, such as whether dose intensity is important in IL-2 therapy (Kopp, W. C., et al., 1994, Cancer Chemotherapy and Biol. Response Modifiers 15:226-286). Numerous reports have documented IL-2 associated toxicity involving increased nitrate levels and the syndrome of vascular leak and hypotension, analogous to septic shock. In addition, an increased incidence of nonopportunistic bacterial infections and autoimmune complications are frequently accompanied by the antitumor response of IL-2 (Rosenberg, 2001, In Cancer Principles and Practice of Oncology, 6^{th} Edition, edited by DeVita et al., Chapter 18, Lippincott, Williams and Wilkins, Philadephia; Kopp, W. C., et al., 1994, Cancer Chemotherapy and Biol. Response Modifiers 15:226-286).

### 2.4.2. Tumor Necrosis Factor

The toxicity of systemically administered TNF seriously limits its use for the treatment of cancer. TNF has been most effective when used for regional therapy, in which measures, such as limb isolation for perfusion, are taken to limit the systemic dose and hence the toxicity of TNF. Dose-limiting toxicity of TNF consist of thrombocytopenia, headache, confusion and hypotension (Mittleman, A., et al., 1992, Inv. New Drugs 10:183-190).

### 2.4.3. Interferons

The activity of IFN-α has been described as being modest in a number of malignancies, including renal cell carcinoma, melanoma, hairy cell leukemia low-grade non-Hodgkin's lymphoma, and others. Higher doses of IFN-α are usually associated with higher response rates in some malignancies, but also cause more toxicity.

### 2.5. Pharmacokinetic Models for Anticancer Chemotherapeutic and Immunotherapeutic. Drugs: Extrapolation and Scaling of Animal Data to Humans

The ethical and fiscal constraints which require the use of animal models for most toxicology research also impose the acceptance of certain fundamental assumptions in order to estimate dose potency in humans from dose-response data in animals. Interspecies dose-response equivalence is most frequently estimated as the product of a reference species dose and a single scaling ratio based on a physiological parameter such as body weight, body surface area, maximum lifespan potential, etc. Most frequently, exposure is expressed as milligrams of dose administered in proportion to body mass in kilograms (mg kg⁻¹). Body mass is a surrogate for body volume, and therefore, the ratio milligrams per kilogram is actually concentrations in milligrams per liter (Hirshaut, Y., et al., 1969, Cancer Res. 29:1732-1740). The key assumptions which accompany this practice and contribute to its failure to accurately estimate equipotent exposure among various species are: i) that the biological systems involved are homogeneous, “well-stirred volumes” with specific gravity equal to 1.0; ii) that the administered compounds are instantly and homogeneously distributed throughout the total body mass; and iii) that the response of the biological systems is directly proportional only to the initial concentration of the test material in the system. As actual pharmacokinetic conditions depart from these assumptions, the utility of initial concentration scaling between species declines.

Through pharmacokinetics, one can study the time course of a drug and its metabolite levels in different fluids, tissues, and excreta of the body, and the mathematical relationships required to develop models to interpret such data. It, therefore, provides the basic information regarding drug distribution, availability, and the resulting toxicity in the tissues and hence, specifies the limitation in the drug dosage for different treatment schedules and different routes of drug administration. The ultimate goal of the pharmacokinetic studies of anticancer and anti-infective drugs is thus to offer a framework for the design of optimal therapeutic dosage regimens and treatment schedules for individual patients.

The currently utilized guidelines for prescription have evolved gradually without always having a complete and explicit justification. For example, in 1966, Freireich and co-workers proposed the use of surface area proportions for interspecies extrapolation of the acute toxicity of anticancer drugs. This procedure has become the method of choice for many risk assessment applications (Freireich, E. J., et al., 1966, Cancer Chemotherapy Rep. 50:219-244). For example, surface area scaling is the basis of the National Cancer Institute's interspecies extrapolation procedure for anti-cancer drugs (Schein, P. S., et al., 1970, Clin. Pharmacol. Therap. 11:3-40; Goldsmith, M. A., et al., 1975, Cancer Res. 35:1354-1364). In accepting surface area extrapolation, the tenuous basis for initial concentration scaling has been replaced by an empirical approach. The basic formula used for estimating prescription of cancer chemotherapy per body surface area (BSA) is BSA=k×kf^{2/3}, in which k is a constant that differs for each age group and species. For example, the k value for adult humans is 11, while for mice it is 9 (See Quiring, P., 1955, Surface area determination, in Glasser E. (ed.) Medical Physics I Chicago: Medical Year Book, p. 1490 and Vriesendorp, H. M., 1985, Hematol. (Supplm. 16) 13:57-63). The major attraction of expressing cancer chemotherapy per m² BSA appears to be that it offers an easily remembered simplification, i.e., equal doses of drug per m² BSA will produce approximately the same effect in comparing different species and age groups. However, simplicity is not proof and alternative methods for estimating prescription of anticancer drugs appear to have a better scientific foundation, with the added potential for a more effective use of anticancer agents (Hill, J. A., et al., 1989, Health Physics 57:395-401).

The effectiveness of an optimal dose of a drug used in chemotherapy and/or immunotherapy can be altered by various factors, including tumor growth kinetics, drug resistance of tumor cells or infectious agents, total-body tumor cell burden, toxic effects of chemotherapy and/or immunotherapy on cells and tissues other than the tumor, and distribution of anti-infective, chemotherapeutic, and/or immunotherapeutic agents within the tissues of the patient. The greater the size of the primary tumor, the greater the probability that a large number of cells (drug resistant and drug sensitive) have metastasized before diagnosis and that the patient will relapse after the primary.

Some metastases arise in certain sites in the body where resistance to chemotherapy is based on the limited tissue distribution of chemotherapeutic drugs administered in standard doses. Such sites act as sanctuaries that shield the cancer cells from drugs that are circulating in the blood; for example, there are barriers in the brain and testes that impede drug diffusion from the capillaries into the tissue. Thus, these sites may require special forms of treatment such as immunotherapy, especially since immunosuppression is characteristic of several types of neoplastic diseases. Similarly, infectious agents, especially intracellular pathogens, may be insulated within environments protected from anti-infective compounds circulating in the blood and, therefore may also required special forms of treatment, including immunotherapy.

### 3. SUMMARY OF THE INVENTION

The methods of the invention comprise methods of eliciting an immune response in an individual in whom the treatment or prevention of cancer or infectious disease is desired by administering a composition comprising an immunogenic amount of unfractionated cellular proteins. In a preferred embodiment, the composition is autologous to the individual; that is, the unfractionated cellular proteins are isolated from the cancer cells of the individual himself (e.g., preferably prepared from tumor biopsies of the patient), or from cells isolated from a metastasis thereof. Alternatively, the composition is allogeneic to the individual, that is, the unfractionated cellular proteins of the composition are prepared from other individuals or from recombinant or non-recombinant cell lines that express one or more antigens of interest.

Specific therapeutic regimens, pharmaceutical compositions, and kits are provided by the invention.

The present invention encompasses methods for prevention and treatment of cancer by enhancing the host's immune competence and activity of immune effector cells. The amounts of the composition as discovered by the present inventor to be effective are surprisingly smaller than those amounts predicted to be effective by extrapolation by prior art methods or from dosages used in animal studies.

Immunotherapy using the therapeutic regimens of the invention, by administering an immunogenic amount of such compositions comprising unfractionated cellular proteins that is effective for such treatment, can induce specific immunity to tumor cells, and leads to regression of the tumor mass. Cancers which are responsive to specific immunotherapy by the compositions comprising unfractionated cellular proteins of the invention include but are not limited to human sarcomas and carcinomas. In a specific embodiment, compositions comprising unfractionated cellular proteins, are allogeneic to the patient; in a preferred embodiment, the compositions comprising unfractionated cellular proteins are autologous to (derived from) the patient to whom they are administered.

Particular compositions of the invention and their properties are described in the sections and subsections which follow. Preferred compositions comprising unfractionated cellular proteins or unfractionated cytosolic soluble proteins, are isolated from tumor biopsy of the patient to whom the composition is to be administered. Such compositions, may be combined with hsp70, hsp90 and/or gp96 complexes, and it would be expected that these compositions would demonstrate strong inhibition of a variety of tumors in mammals. Moreover, the therapeutic doses of the compositions comprising unfractionated cellular proteins, that are effective in the corresponding experimental model in rodents as described infra, in Section 8 can be used to inhibit the in vivo growth of colon and liver cancers in human cancer patients as described in Sections 6 and 7, infra. Preferred compositions which preferably exhibit no toxicity when administered to human subjects are also described.

In another embodiment, the methods further optionally comprise administering biological response modifiers, e.g., IFN-α, IFN-γ, IL-2, IL-4, IL-6, TNF, or other cytokine growth factors affecting the immune cells, in combination with compositions comprising unfractionated cellular proteins.

In addition to cancer therapy, compositions comprising unfractionated cellular proteins can be utilized for the prevention of a variety of cancers, e.g., in individuals who are predisposed as a result of familial history or in individuals with an enhanced risk to cancer due to environmental factors.

The invention provides a method of inducing an immune response in a subject against a type of cancer which comprises administering to the subject a composition comprising an immunogenic amount of unfractionated cellular proteins obtained from cells of said type of cancer or a metastasis thereof effective to induce said immune response. The invention also provides a method of treating or preventing a type of cancer, comprising administering to a subject in need of such treatment or prevention a composition comprising an amount, effective for treatment or prevention, of unfractionated cellular proteins obtained from cells of the type of cancer or a metastasis thereof.

In specific embodiments, the disclosed methods for inducing an immune response and for preventing or treating a type of cancer are carried out by administering unfractionated cellular proteins from 10² to 10⁹ cell equivalents of cells from the target type of cancer or metastasis thereof, from 10² to 10⁷ cell equivalents of cells from the target type of cancer or metastasis thereof, and, preferably, proteins from less than 10⁶ cell equivalents, from 10² to 5×10⁵ cell equivalents of cells. In a preferred embodiment, the amount of unfractionated cellular proteins administered are in the range of proteins of 10³ cell equivalents or less of the cells, in a more preferred embodiment from 10⁴ cell equivalents or less, and in a even more preferred embodiment, proteins of 5×10⁵ cell equivalents or less.

The unfractionated cellular proteins used in the disclosed methods for inducing an immune response and for preventing or treating a type of cancer, are preferably prepared by a method comprising subjecting a lysed sample of said cells to centrifugation one or more times with the highest force being about 100,000×g, and substantially not subjecting the proteins within the lysed sample to any method that selectively removes particular soluble proteins. The unfractionated cellular proteins so prepared, are contained in a solution substantially free of plasma membrane, cell organelles or particles thereof, and viral particles.

In a specific embodiment, the proteins used in the disclosed methods for inducing an immune response and for preventing or treating a type of cancer, are prepared by a method comprising subjecting a lysed sample of said cells to centrifugation one or more times with the highest force being 1,000×g, and substantially not subjecting the proteins within the lysed sample to any method that selectively removes particular proteins. The unfractionated cellular proteins so prepared, are contained in a solution substantially free of intact cells.

In one embodiment of the disclosed methods for inducing an immune response and for preventing or treating a type of cancer, the proteins are autologous to the subject. In another embodiment, the proteins are allogeneic to the subject.

In other embodiments, the unfractionated cellular proteins are isolated from cells obtained from a tumor or from cells of a tumor cell line.

In another embodiment of the disclosed methods for inducing an immune response and for preventing or treating a type of cancer, the composition administered further comprises an adjuvant. Alternatively, the composition administered is substantially free of adjuvant.

In still another embodiment of the disclosed methods for inducing an immune response and for preventing or treating a type of cancer, the composition is administered at weekly intervals. This administration, in one embodiment, is repeated at the same site of the subject. Alternatively, the administration is repeated at different sites. The composition may be administered intradermally, or subcutaneously.

In specific embodiments of the disclosed methods for preventing or treating a type of cancer, the type of cancer is a sarcoma or carcinoma, selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenström's macroglobulinemia, and heavy chain disease.

Also disclosed herein is a method of inducing an immune response in a subject against a pathogen, which comprises administering to the subject a composition comprising an immunogenic amount of unfractionated cellular proteins obtained from cells having an antigenicity (antigenic determinant) of said pathogen. A further method disclosed herein is for treating or preventing an infection by a pathogen in a subject comprising administering to a subject in need of such treatment or prevention a composition comprising an amount, effective for such treatment or prevention, of unfractionated cellular proteins obtained from cells having an antigenicity of said pathogen.

In one embodiment of the disclosed methods for inducing an immune response against, and for preventing or treating an infection by a pathogen in a subject, the proteins are obtained from cells infected with an agent displaying the antigenicity of said pathogen or from cells transformed with and expressing a nucleic acid displaying the antigenicity of said pathogen.

In a specific embodiment, the disclosed methods for inducing an immune response against, and for preventing or treating an infection by a pathogen in a subject, are carried out by administering unfractionated cellular proteins from 10² to 10⁷ cell equivalents of cells having an antigenicity of the pathogen, from 10² to 10⁶ cell equivalents of cells having an antigenicity of the pathogen, and, preferably, proteins from less than 10⁶ cell equivalents, or from 10² to 5×10⁵ cell equivalents of cells. In a preferred embodiment, the proteins are isolated from 10³ cell equivalents or less of the cells, in a more preferred embodiment from 10⁴ cell equivalents or less, and in a even more preferred embodiment, the proteins are isolated from 5×10⁵ cell equivalents or less.

The proteins used in the disclosed methods for inducing an immune response against, and for preventing or treating an infection by a pathogen in a subject, are prepared by a method comprising subjecting a lysed sample of said cells to centrifugation one or more times with the highest force being 100,000×g, and substantially not subjecting cytosolic, soluble proteins within the lysed sample to any method that selectively removes particular soluble proteins. The unfractionated cellular proteins so prepared, are contained in a solution substantially free of plasma membrane, and cell organelles or particles thereof.

In a further embodiment, the proteins used in the disclosed methods for inducing an immune response against, and for preventing or treating an infection by a pathogen in a subject, are prepared by a method comprising subjecting a lysed sample of said cells to centrifugation one or more times with the highest force being 1,000×g, and substantially not subjecting the proteins within the lysed sample to any method that selectively removes particular soluble proteins. The unfractionated cellular proteins so prepared, are contained in a solution substantially free of intact cells.

In another embodiment of the disclosed methods for inducing an immune response against, and for preventing or treating an infection by a pathogen in a subject, the proteins are autologous to the subject. In another embodiment, the proteins are allogeneic to the subject.

In other embodiments, the unfractionated cellular proteins are isolated from cells infected with the pathogen, from cells infected with replication-defective or other impaired or attenuated derivatives of the pathogen, or from cells transformed with recombinant molecules expressing an antigenicity of the pathogen.

In another embodiment of the disclosed methods for inducing an immune response against, and for preventing or treating an infection by a pathogen in a subject, the composition administered further comprises an adjuvant. Alternatively, the composition administered is substantially free of adjuvant.

In still another embodiment of the disclosed methods for inducing an immune response against, and for preventing or treating an infection by a pathogen in a subject, the composition is administered at weekly intervals. This administration, in one embodiment, is repeated at the same site of the subject. Alternatively, the administration is repeated at different sites. The composition may be administered intradermally, or subcutaneously.

The disclosed methods are used, in a further embodiment, for inducing an immune response against, and for preventing or treating an infection by a pathogen in a subject, where that pathogen is a virus, bacterium, or a parasite; that is, the disclosed methods may be used where the pathogen is selected from the group consisting of hepatitis virus type A, hepatitis virus type B, hepatitis virus type C, influenza virus, varicella virus, adenovirus, herpes simplex virus type I (HSV-I), herpes simplex virus type II (HSV-II), rinderpest virus, rhinovirus, echovirus, rotavirus, respiratory syncytial virus, papilloma virus, papova virus, cytomegalovirus, echinovirus, arbovirus, hantavirus, coxsackie virus, mumps virus, measles virus, rubella virus, polio virus, human immunodeficiency virus type I (HIV-I), human immunodeficiency virus type II (HIV-II), mycobacteria rickettsia, mycoplasma, neisseria, legionella, leishmania, kokzidioa, trypanosoma, chlamydia and rickettsia.

In a further embodiment of the disclosed methods for inducing an immune response against, and for preventing or treating an infection by a pathogen in a subject, the administered composition further comprises at least one biological response modifier selected from the group comprising heat shock proteins, interferons, interleukins, colony stimulating factors, monoclonal antibodies, and tumor necrosis factor.

The invention further provides a method for preparing a vaccine for treatment or prevention of cancer comprising the steps of lysing cancer cells to produce a crude cell lysate; centrifuging the crude cell lysate or supernatant derived therefrom one or more times, to remove intact cells, cell membranes, and organelles, whereby there is substantially no subjecting of cellular proteins within said lysate to any method that selectively removes particular soluble proteins. In a further embodiment, the lysing step is done by using hypotonic shock combined with mechanical disruption. In a further embodiment, the lysing step is carried out by suspending cells in a buffer that can, but need not be a hypotonic buffer, and subjecting the suspension to repeated cycles of freezing and thawing. In other aspects of this embodiment, resuspended cells are lysed by sonication or Dounce homogenization. In yet another embodiment of this method, the centrifuging step comprises a first centrifuging at 1,000×g to produce a first supernatant, and a second centrifuging at 100,000×g of said first supernatant to produce a second supernatant. In addition, the second supernatant is dialyzed against a suitable buffer.

In yet another embodiment of this method, the centrifuging step comprises centrifuging the cell lysate only at 1,000×g to produce a supernatant that is then dialyzed against a suitable buffer.

The present invention also encompasses a method of treating or preventing a type of cancer, comprising administering to a subject in need of such treatment or prevention a composition comprising an amount, effective for the treatment or prevention, of unfractionated cellular proteins obtained from cells transformed with and expressing a nucleic acid encoding a molecule displaying antigenicity of a tumor-associated antigen or tumor-specific antigen of said type of cancer.

Another embodiment of the present invention includes a kit comprising in one or more containers an amount, effective for treatment or prevention of a type of cancer, of unfractionated cellular proteins obtained from cells of said type of cancer or a metastasis thereof or from cells transformed with and expressing a nucleic acid encoding a molecule displaying antigenicity of a tumor-associated antigen or tumor-specific antigen of said type of cancer.

A further embodiment of the present invention includes a kit comprising in one or more containers an amount, effective for treatment or prevention of an infectious disease, of unfractionated cellular proteins obtained from cells having an antigenicity of a pathogen that causes the infectious disease. In a further embodiment of a kit comprising unfractionated cytoplasmic soluble proteins effective for the treatment or prevention of an infectious disease, the proteins are obtained from cells infected with an agent displaying the antigenicity of said pathogen or from cells transformed with and expressing a nucleic acid displaying the antigenicity of said pathogen.

The Examples presented in Sections 6, 7 and 8 below, detail the use according to the methods of the invention of compositions comprising unfractionated cellular proteins in cancer immunotherapy in experimental tumor models and in human patients suffering from advanced colon and liver cancer.

### 4. DETAILED DESCRIPTION OF THE INVENTION

Methods and compositions for the prevention and treatment of primary and metastatic neoplastic diseases and infectious diseases and for eliciting an immune response in a human individual, are described. The invention involves administration of compositions comprising unfractionated cellular proteins.

The term “unfractionated cellular proteins” as used herein, means a collection of proteins contained within a clarified extract of lysed cells, wherein the clarified extract is not subjected to any methods that selectively remove particular proteins, and substantially lacks intact cells.

The term “unfractionated cytosolic soluble proteins” refers to the collection of proteins contained within a clarified extract of lysed cells, wherein the clarified extract is not subjected to any methods that selectively remove particular soluble proteins, and substantially lacks not only intact cells, but also cell debris, nuclei, organelles and membranes.

The term “unfractionated cellular proteins” as used herein also encompasses “unfractionated cytosolic soluble proteins.”

In one embodiment, a clarified cell extract can be prepared by cell lysis and low-speed centrifugation. The low-speed centrifugation is designed to remove intact cells. The clarified cell extract comprises unfractionated cellular proteins, including proteins associated with organelles, and cellular membranes, such as plasma membranes and membranes of the endoplasmic reticulum. The clarified cell extract may be dialyzed against a suitable buffer solution before use.

In another embodiment, after the low-speed centrifugation, the clarified cell extract is subjected to a high-speed centrifugation. The high-speed centrifugation is generally at 100,000×g for one hour, which is sufficient to remove most of the remaining cell debris, nuclei, organelles and cellular membranes. The supernatant collected after this step comprises unfractionated cytosolic soluble proteins. The supernatant may be dialyzed against a suitable buffer solution, but it is not subject to any additional chromatographic separations.

The compositions comprising unfractionated cellular proteins or unfractionated cytosolic soluble proteins are administered to elicit an effective specific immune response.

### 4.1. Unfractionated Cellular Proteins

The methods of the invention comprise methods of eliciting an immune response in an individual or in whom the treatment or prevention of infectious diseases or cancer is desired by administering a composition comprising unfractionated cellular proteins. The unfractionated cellular proteins which include unfractionated cytosolic soluble proteins can be obtained from non-recombinant cells (containing endogenous heat shock protein-peptide complexes) or recombinant cells containing non-endogenous heat shock protein-peptide complexes. For example, in a preferred embodiment for the treatment of cancer, the unfractionated cellular proteins are prepared, postoperatively, from tumor cells obtained from the cancer patient.

In another embodiment of the present invention, proteins, protein fragments, and peptide antigens of interest are synthesized in cell lines modified by the introduction of recombinant expression systems that encode such antigens, and such cells are used to prepare the unfractionated cellular proteins. Suitable proteins and peptides that may be expressed in such cells include, but are not limited to those displaying the antigenicity of (for the treatment or prevention of cancer) tumor antigens including tyrosinase, gp100, melan-A, gp75, mucins; and (for the treatment or prevention of infectious disease): viral proteins including proteins of immunodeficiency virus type I (HIV-I), human immunodeficiency virus type II (HIV-II), hepatitis type A, hepatitis type B, hepatitis type C, influenza, Varicella, adenovirus, herpes simplex type I (HSV-I), herpes simplex type II (HSV-II), rinderpest, rhinovirus, echovirus,. rotavirus, respiratory syncytial virus, papilloma virus, papova virus, cytomegalovirus, echinovirus, arbovirus, hantavirus, coxsackie virus, mumps virus, measles virus, rubella virus and polio virus, as well as proteins or protein fragments of infectious agents including, but not limited to, mycobacteria rickettsia, mycoplasma, neisseria, legionella, leishmania, kokzidioa, trypanosoma, chlamydia and rickettsia.

In a preferred embodiment, the unfractionated cellular proteins including unfractionated cytosolic soluble proteins are autologous to the individual; that is, the unfractionated cytosolic soluble proteins are isolated from either from the infected cells or the cancer cells or precancerous cells of the individual himself (e.g., preferably prepared from infected tissues or tumor biopsies of the patient).. Alternatively, in another embodiment of the present invention, the unfractionated cellular proteins are allogeneic to the treated individual, that is, the proteins are prepared from an individual other than the patient to whom they are administered. Unfractionated cellular proteins are also prepared from cell lines produced in vitro by recombinant methods whereby the modified cells express an exogenous antigenic molecule of interest, or an increased level of an endogenous antigenic molecule of interest. Such exogenous antigens, and fragments or derivatives thereof can be selected from among those known in the art, as well as those readily identified by standard immunoassays known in the art by the ability to bind antibody or MHC molecules (antigenicity) or generate immune response (immunogenicity). Unfractionated cytosolic soluble proteins can be prepared from cancerous or precancerous tissue of a patient, or from a cancer cell line, or can be produced from cell lines constructed by recombinant methods, in vitro, and which express one or more antigenic molecules of interest.

The unfractionated cellular proteins of the present invention can be used alone or may be combined with heat shock proteins, including but not limited to, hsp70, hsp90, gp96 alone or combinations thereof, preferably complexed noncovalently or covalently with an antigenic molecule. Preferably, the heat shock proteins are human heat shock proteins.

Heat shock proteins, which are also referred to interchangeably herein as stress proteins, useful in the practice of the instant invention can be selected from among any cellular protein that satisfies the following criteria. It is a protein whose intracellular concentration increases when a cell is exposed to a stressful stimulus, it is capable of binding other proteins or peptides, it is capable of releasing the bound proteins or peptides in the presence of adenosine triphosphate (ATP) or low pH, or it is a protein showing at least 35% homology with any cellular protein having any of the above properties.

The first stress proteins to be identified were the heat shock proteins (hsps). As their name implies, hsps are synthesized by a cell in response to heat shock. To date, three major families of hsp have been identified based on molecular weight. The families have been called hsp60, hsp70 and hsp90 where the numbers reflect the approximate molecular weight of the stress proteins in kilodaltons. Many members of these families were found subsequently to be induced in response to other stressful stimuli including, but not limited to, nutrient deprivation, metabolic disruption, oxygen radicals, and infection with intracellular pathogens. (See Welch, May 1993, Scientific American 56-64; Young, 1990, Annu. Rev. Immunol. 8:401-420; Craig, 1993, Science 260:1902-1903; Gething, et al., 1992, Nature 355:33-45; and Lindquist, et al., 1988, Annu. Rev. Genetics 22:631-677), the disclosures of which are incorporated herein by reference. It is contemplated that hsps/stress proteins belonging to all of these three families can be combined with the compositions of the present invention comprising unfractionated cellular proteins, in the practice of the instant invention.

The major hsps can accumulate to very high levels in stressed cells, but they occur at low to moderate levels in cells that have not been stressed. For example, the highly inducible mammalian hsp70 is hardly detectable at normal temperatures but becomes one of the most actively synthesized proteins in the cell upon heat shock (Welch, et al., 1985, J. Cell. Biol. 101:1198-1211). In contrast, hsp90 and hsp60 proteins are abundant at normal temperatures in most, but not all, mammalian cells and are further induced by heat (Lai, et al., 1984, Mol. Cell. Biol. 4:2802-10; van Bergen en Henegouwen, et al., 1987, Genes Dev. 1:525-31).

Heat shock proteins are among the most highly conserved proteins in existence. For example, DnaK, the hsp70 from E. coli has about 50% amino acid sequence identity with hsp70 proteins from excoriates (Bardwell, et al., 1984, Proc. Natl. Acad. Sci. 81:848-852). The hsp60 and hsp90 families also show similarly high levels of intra families conservation (Hickey, et al., 1989, Mol. Cell. Biol. 9:2615-2626; Jindal, .1989, Mol. Cell. Biol. 9:2279-2283). In addition, it has been discovered that the hsp60, hsp70 and hsp90 families are composed of proteins that are related to the stress proteins in sequence, for example, having greater than 35% amino acid identity, but whose expression levels are not altered by stress. Therefore it is contemplated that the definition of stress protein, as used herein, embraces other proteins, muteins, analogs, and variants thereof having at least 35% to 55%, preferably 55% to 75%, and most preferably 75% to 85% amino acid identity with members of the three families whose expression levels in a cell are enhanced in response to a stressful stimulus. The purification of stress proteins belonging to these three families is described below.

The immunogenic hsp-peptide complexes that may be combined with unfractionated cellular proteins of the invention, may include any complex containing an hsp and a peptide that is capable of inducing an immune response in a mammal. The peptides are preferably non covalently associated with the hsp. Preferred complexes may include, but are not limited to, hsp60-peptide, hsp70-peptide and hsp90-peptide complexes. For example, a complex of an antigenic molecule and an hsp called gp96 which is present in the endoplasmic reticulum of eukaryotic cells and is related to the cytoplasmic hsp90's can be combined with the compositions of the present invention comprising unfractionated cellular proteins, to generate an effective vaccine.

Although the compositions comprising unfractionated cellular proteins can be allogeneic to the patient, in a preferred embodiment they are autologous to (derived from) the patient to whom they are administered. The invention provides methods for determining doses for human cancer immunotherapy by evaluating the optimal dose of compositions comprising unfractionated cellular proteins or unfractionated cytosolic soluble proteins in experimental tumor models.

The invention provides compositions which enhance the immunocompetence of the host individual and elicit specific immunity against infectious agents or specific immunity against preneoplastic and neoplastic cells. The therapeutic regimens and pharmaceutical compositions of the invention are described below. These compositions have the capacity to prevent the onset and progression of infectious diseases and prevent the development of tumor cells and to inhibit the growth and progression of tumor cells indicating that such compositions can induce specific immunity in infectious diseases and cancer immunotherapy.

Compositions comprising unfractionated cellular proteins appear to induce an inflammatory reaction at the tumor site and ultimately may cause a regression of the tumor burden in the cancer patients treated. Cancers which can be treated with compositions comprising unfractionated cellular proteins, include, but are not limited to, human sarcomas and carcinomas. In a particular embodiment, the unfractionated cellular proteins are unfractionated cytosolic soluble proteins.

In another embodiment, the compositions of the present invention, comprising an immunogenic, effective amount of unfractionated cellular proteins obtained from cancer cells or a metastasis thereof, are administered to a subject in need of treatment against cancer, as a method of inducing an immune response against that cancer. In this method, the proteins administered are preferably from 10⁷ cell equivalents or less, from 10⁶ cell equivalents or less, or from 10² to 5×10⁵ cell equivalents, or from 10³ cell equivalents or less, more preferably from 10⁴ cell equivalents or less, and most preferably from 5×10⁵ cell equivalents or less. Accordingly, the invention provides methods of preventing and treating cancer in an individual comprising administering a composition which stimulates the immunocompetence of the host individual and elicits specific immunity against the preneoplastic and/or neoplastic cells. As used herein, “preneoplastic” cell refers to a cell which is in transition from a normal to a neoplastic form; and morphological evidence, increasingly supported by molecular biologic studies, indicates that preneoplasia progresses through multiple steps. Non-neoplastic cell growth commonly consists of hyperplasia, metaplasia, or most particularly, dysplasia (for review of such abnormal growth conditions (See Robbins and Angell, 1976, Basic Pathology, 2d Ed., W. B. Saunders Co., Philadelphia, pp. 68-79). Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. Although preneoplastic lesions may progress to neoplasia, they may also remain stable for long periods and may even regress, particularly if the inciting agent is removed or if the lesion succumbs to an immunological attack by its host.

The therapeutic regimens and pharmaceutical compositions of the invention that comprise unfractionated cellular proteins, may be used in conjunction with additional immune response enhancers or biological response modifiers including, but not limited to, the cytokines IFN-α, IFN-γ, IL-2, IL-4, IL-6, TNF, or other cytokine affecting immune cells, as well as complexes of heat shock proteins and antigenic molecules. Furthermore, the compositions of the present invention may be administered either with, or in a preferred embodiment, without an adjuvant.

The invention further relates to administration of compositions comprising unfractionated cellular proteins to individuals at enhanced risk of cancer, e.g., due to familial history or environmental risk factors.

### 4.1.1. Preparation of Unfractionated Cellular Proteins

An exemplary, but not limiting, method that may be used to prepare unfractionated cellular proteins is as follows:

Cells, which may be tumor cells derived from a biopsy of the patient or tumor cells cultivated in vitro, or cells lines infected with a pathogenic agent, are suspended in 3 volumes of 1× Lysis buffer consisting of 30 mM sodium bicarbonate pH 7.5, and 1 mM phenyl methyl sulfonyl fluoride (PMSF). The cells may be lysed by mechanical shearing in the same Lysis buffer, which are incubated on ice for about 20 minutes to allow the cells to become hypotonically-swollen, and which are then homogenized in a dounce homogenizer until >95% cells are lysed. In other embodiments, cells resuspended in a non-hypotonic buffer, such as PBS, are lysed by cycles of freezing and thawing, or sonication. For example, two to five, and preferably three, such cycles of freezing and thawing are used, as necessary, generally until at least 90% of the cells have been lysed. For sonication, cells in PBS and on ice can be sonicated using a Ultrasonic Processor GE130 for 5 cycles; each cycle consisting of 10 seconds of exposure to ultrasound and thirty seconds of rest before the next cycle of sonication.

The lysate can be centrifuged one or more times at a maximum of about 1,000×g for a period of time, e.g., 10 minutes, to remove unbroken cells, or until the lysate becomes classified. The clarified cell extract which comprises unfractionated cellular proteins can be dialyzed, generally for 36 hours at 4° C. (three times, 100 volumes each time) against PBS (phosphate buffered saline) or other suitable buffer, to provide the unfractionated cellular proteins of the present invention. If necessary, insoluble material in the cell extract may be removed by filtration or further low-speed centrifugation.

### 4.1.2. Preparation of Unfractionated Cytosolic Soluble Proteins

An exemplary, but not limiting, method that may be used to prepare unfractionated cytosolic soluble proteins is as follows:

The clarified cell extract which comprises unfractionated cellular proteins prepared as described in Section 4.1.1 is recentrifuged for one or more times at about 100,000×g for a period of time, for example, about one hour, or until the supernatant is substantially free of plasma membranes, cell organelles or particles there of, and viral particles. A higher speed centrifugation can also be used, e.g., greater than or about 125,000×g, 150,000×g, 175,000×g or 200,000×g. The recovered supernatant comprises unfractionated cytosolic soluble proteins of the present invention and may be dialyzed for 36 hours at 4° (three times, 100 volumes each time) against PBS (phosphate buffered saline) or other suitable buffer. If necessary, any remaining insoluble material in the preparation may be removed by filtration or further centrifugation.

### 4.1.3. Exogenous Antigenic Molecules

In one embodiment of the present invention, unfractionated cellular proteins are isolated from cells transformed with nucleic acids encoding and expressing one or more tumor-associated antigens or tumor-specific antigens (for the treatment or prevention of cancer), or expressing one or more molecules that display the antigenicity of a pathogen (for the treatment or prevention of infectious disease). Specific antigens or antigenic portions thereof that can be used as antigenic molecules to be expressed in cell lines from which the unfractionated cytosolic soluble proteins of the present invention may be isolated, are selected from among those known in the art or determined by immunoassay to be able to bind to antibody or MHC molecules (antigenicity) or generate an immune response (immunogenicity). To determine immunogenicity or antigenicity by detecting binding to antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in vivo immunoassays (using colloidal gold, enzyme or radioisotope labels, for example), western blots, immunoprecipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are envisioned for use. In one embodiment for detecting immunogenicity, T cell-mediated responses can be assayed by standard methods, e.g., in vitro cytotoxicity assays or in vivo delayed-type hypersensitivity assays.

Potentially useful antigens or derivatives thereof for use as antigenic molecules can also be identified by various criteria, such as the antigen's involvement in neutralization of a pathogen's infectivity (wherein it is desired to treat or prevent infection by such a pathogen) (Norrby, 1985, Summary, in Vaccines 85, Lerner, et al. (eds.), Cold Spring Harbor Laboratory, Cold Spring Harbor, N.Y., pp. 388-389), type or group specificity, recognition by patients' antisera or immune cells, and/or the demonstration of protective effects of antisera or immune cells specific for the antigen. In addition, where it is desired to treat or prevent a disease caused by pathogen, the antigen's encoded epitope should preferably display a small or no degree of antigenic variation in time or amongst different isolates of the same pathogen.

Preferably, where it is desired to treat or prevent cancer, known tumor-specific antigens or fragments or derivatives thereof are used. For example, such tumor specific or tumor-associated antigens include but are not limited to KS 1/4 pan-carcinoma antigen (Perez and Walker, 1990, J. Immunol. 142:3662-3667; Bumal, 1988, Hybridoma 7(4) :407-415); ovarian carcinoma antigen (CA125) (Yu, et al., 1991, Cancer Res. 51(2):468-475); prostatic acid phosphate (Tailer, et al., 1990, Nucl. Acids Res. 18(16):4928); prostate specific antigen (Henttu and Vihko, 1989, Biochem. Biophys. Res. Comm. 160(2) :903-910; Israeli, et al., 1993, Cancer Res. 53:227-230); melanoma-associated antigen p97 (Estin, et al., 1989, J. Natl. Cancer Inst. 81(6):445-446); melanoma antigen gp75 (Vijayasardahl, et al., 1990, J. Exp. Med. 171(4) :1375-1380); high molecular weight melanoma antigen (Natali, et al., 1987, Cancer 59:55-63) and prostate specific membrane antigen.

In a specific embodiment, an antigen or fragment or derivative thereof specific to a certain tumor is selected for expression in a cell line to be used for the preparation of unfractionated cellular proteins that are subsequently administered to a patient having that tumor.

Preferably, where it is desired to treat or prevent viral diseases, molecules comprising epitopes of known viruses are expressed in a cell line to be used for the preparation unfractionated cytosolic soluble proteins. For example, such antigenic epitopes may be prepared from viruses including, but not limited to, hepatitis type A hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II). Preferably, where it is desired to treat or prevent bacterial infections, molecules comprising epitopes of known bacteria are used. For example, such antigenic epitopes may be prepared from bacteria including, but not limited to, mycobacteria rickettsia, mycoplasma, neisseria and legionella.

Where it is desired to treat or prevent protozoal infections, molecules comprising epitopes of known protozoa are expressed in recombinant cells lines from which unfractionated cytosolic soluble proteins are prepared. For example, such antigenic epitopes may be prepared from protozoa including, but not limited to, leishmania, kokzidioa, and trypanosoma.

Preferably, where it is desired to treat or prevent parasitic infectious, molecules comprising epitopes of known parasites are expressed in recombinant cells lines from which unfractionated cellular proteins are prepared. For example, such antigenic epitopes may be from parasites including, but not limited to, chlamydia and rickettsia.

### 4.1.4. Determination of Immunogenicity of Unfractionated Cellular Proteins

The unfractionated cellular proteins prepared as described herein can be assayed for immunogenicity using the mixed lymphocyte target culture assay (MLTC) well known in the art.

By way of example but not limitation, the following procedure can be used. Briefly, mice are injected subcutaneously with the candidate unfractionated cytosolic soluble proteins, while other mice are injected with either another composition comprising unfractionated cytosolic soluble proteins, whole tumor cells or whole infected cells which act as positive controls for the assay. The mice are injected twice, 7-10 days apart. Ten days after the last immunization, spleens are removed and lymphocytes released. The released lymphocytes may be restimulated subsequently in vitro by the addition of dead cells that expressed the complex of interest.

For example, 8×10⁶ immune spleen cells may be stimulated with 4×10⁴ mitomycin C treated or γ-irradiated (5-10,000 rads) infected cells (or tumor cells, or cells transfected with an appropriate gene, as the case may be) in 3 ml RPMI medium containing 10% fetal calf serum. In certain cases 33% secondary mixed lymphocyte culture supernatant may be included in the culture medium as a source of T cell growth factors (See, Glasebrook, et al., 1980, J. Exp. Med. 151:876). To test the primary cytotoxic T cell response after immunization, spleen cells may be cultured without stimulation. In some experiments spleen cells of the immunized mice may also be restimulated with antigenically distinct cells, to determine the specificity of the cytotoxic T cell response.

Six days later the cultures are tested for cytotoxicity in a 4 hour ⁵¹Cr-release assay (See, Palladino, et al., 1987, Cancer Res. 47:5074-5079 and Blachere, et al., 1993, J. Immunotherapy 14:352-356). In this assay, the mixed lymphocyte culture is added to a target cell suspension to give different effector:target (E:T) ratios (usually 1:1 to 40:1). The target cells are prelabeled by incubating 1×10⁶ target cells in culture medium containing 200 mCi ⁵¹Cr/ml for one hour at 37° C. The cells are washed three times following labeling. Each assay point (E:T ratio) is performed in triplicate and the appropriate controls incorporated to measure spontaneous ⁵¹Cr release (no lymphocytes added to assay) and 100% release (cells lysed with detergent). After incubating the cell mixtures for 4 hours, the cells are peletted by centrifugation at 200 g for 5 minutes. The amount of ⁵¹Cr released into the supernatant is measured by a gamma counter. The percent cytotoxicity is measured as cpm in the test sample minus spontaneously released cpm divided by the total detergent released cpm minus spontaneously released cpm.

In order to block the MHC class I cascade, a concentrated hybridoma supernatant derived from K-44 hybridoma cells (an anti-MHC class I hybridoma) is added to the test samples to a final concentration of 12.5%.

### 4.1.5. Formulation and Administration

Unfractionated cellular proteins, including unfractionated cytosolic soluble proteins of the invention may be formulated into pharmaceutical preparations for administration to animals for inducing an immune response or for treatment or prevention of cancer or infectious diseases. The subject to which compositions comprising unfractionated cytosolic soluble proteins effective in such treatment or prevention may be administered, can be an animal. More specifically, the subject may be a domestic animal, such as a cat, dog, horse, cow, chicken, mouse, rat, etc., or, preferably, a mammal or primate, and, most preferably, a human. Compositions comprising unfractionated cytosolic soluble proteins of the invention formulated in a compatible pharmaceutical carrier may be prepared, packaged, and labeled for treatment of the indicated tumor, such as human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenström's macroglobulinemia, and heavy chain disease. Alternatively, it can be labeled for treatment of the appropriate infectious disease. Alternatively, pharmaceutical compositions may be formulated for treatment of appropriate infectious diseases.

In various embodiments, the administered dosage of unfractionated cellular proteins or unfractionated cytosolic, soluble proteins is 10⁷, 5×10⁶, 10⁶, 5×10⁵, 10⁵, 5×10⁴, 10⁴, 5×10³, 10³, 5×10² or 10² cell equivalents or less, or from 10² to 10⁷ cell equivalents, from 10² to 5×10⁶ cell equivalents, from 10² to 10⁶ cell equivalents, from 10² to 5×10⁵ cell equivalents, from 10² to 10⁵ cell equivalents, from 10² to 5×10⁴ cell equivalents, from 10² to 10⁴ cell equivalents, from 10² to 5×10³ cell equivalents, from 10² to 10³ cell equivalents, and from 10² to 5×10² cell equivalents. Cell equivalents can be determined by comparing the protein content of a sample comprising unfractionated cellular proteins or unfractionated cytosolic soluble proteins to the total protein recovered in the low-speed, or high-speed supernatant fraction, depending upon the method used, prepared from a known number of cells.

If the composition comprising the unfractionated cellular proteins of the present invention is water-soluble, then it may be formulated in an appropriate buffer, for example, phosphate buffered saline or other physiologically compatible solutions. Alternatively, it may be formulated with a non-ionic surfactant such as Tween, or polyethylene glycol. Thus, the compositions and their physiologically acceptable solvates may be formulated for administration by inhalation or insufflation (either through the mouth or the nose) or oral, buccal, parenteral, rectal administration or, in the case of tumors, directly injected into a solid tumor.

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Preparations for oral administration may be suitably formulated to give controlled release of the active compound.

For buccal administration, the compositions may take the form of tablets or lozenges formulated in conventional manner.

For administration by inhalation, compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the composition and a suitable powder base such as lactose or starch.

The compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The pharmaceutical compositions of the invention may further comprise one or more cytokines, complexes of heat shock proteins and antigenic molecules, and/or adjuvants.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers therapeutically or prophylactically effective amounts of compositions comprising unfractionated cellular proteins, in pharmaceutically acceptable form. The compositions comprising unfractionated cellular proteins in a vial of a kit of the invention may be in the form of a pharmaceutically acceptable solution, e.g., in combination with sterile saline, dextrose solution, or buffered solution, or other pharmaceutically acceptable sterile fluid. Alternatively, the composition comprising unfractionated cytosolic soluble proteins, may be lyophilized or desiccated; in this instance, the kit optionally further comprises in a container a pharmaceutically acceptable solution (e.g., saline, dextrose solution, etc.), preferably sterile, to reconstitute the composition to form a solution for injection purposes.

In various embodiments, the unfractionated cellular proteins complexes in the kits are unfractionated cytosolic soluble proteins.

In another embodiment, a kit of the invention further comprises a needle or syringe, preferably packaged in sterile form, for injecting the complex, and/or a packaged alcohol pad. Instructions are optionally included for administration of hsp-antigenic molecule complexes by a clinician or by the patient.

### 4.2. Combination Therapy with Complexes of Heat Shock Proteins and Antigenic Molecules

As an optional procedure, the unfractionated cellular proteins can be administered in combination with complexes of heat shock proteins and antigenic molecules, for eliciting a specific immune response or for treatment or prevention of cancer or infectious disease (e.g., where the antigenic molecule displays the antigenicity of a cancer cell or infectious agent, respectively). An “antigenic molecule” that is complexed to a heat shock protein refers to the peptides with which heat shock proteins are endogenously associated in vivo (e.g., in infected cells or precancerous or cancerous tissue), as well as exogenous antigens/immunogens (i.e. with which the hsps are not naturally complexed in vivo) or antigenic/immunogenic fragments and derivatives thereof.

In one embodiment, the peptides are noncovalently complexed to hsps in vivo, and the complexes can be isolated from cells; or alternatively, complexes of hsps and antigenic molecules can be produced in vitro from purified preparations each of hsps and antigenic molecules, and then combined with the unfractionated cellular proteins.

Thus, in one embodiment, the compositions of the present invention may also comprise immunogenic or antigenic peptides that are endogenously complexed to hsps or MHC antigens. For the treatment or prevention of cancer, such peptides may be prepared that stimulate cytotoxic T cell responses against by displaying the antigenicity of a tumor antigen (e.g., tyrosinase, gp100, melan-A, gp75, mucins, etc.). For the treatment or prevention of infectious disease, such peptides may display the antigenicity of a protein of an infectious agent, e.g., a viral protein including, but not limited to, a protein of immunodeficiency virus type I (HIV-I), human immunodeficiency virus type II (HIV-II), hepatitis type A, hepatitis type B, hepatitis type C, influenza, Varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, hantavirus, coxsackie virus, mumps virus, measles virus, rubella virus, or polio virus.

In another specific embodiment, antigens of cancers (e.g., tumors) or infectious agents (e.g., viral antigen, bacterial antigens, etc.) can be obtained by purification from natural sources, by chemical synthesis, or recombinantly, and, through in vitro procedures such as those described below, noncovalently complexed to hsps that are then combined with the unfractionated cellular proteins.

In an embodiment wherein the hsp-antigenic molecule complex, combined with the unfractionated cellular proteins of the invention is a complex produced in vivo in cells, exemplary purification procedures for those complexes are described in U.S. Pat. No. 5,837,251. Alternatively, in an embodiment wherein one wishes to use antigenic molecules by complexing to hsps in vitro, hsps can be purified for such use from the endogenous hsp-peptide complexes in the presence of ATP or low pH (or chemically synthesized or recombinantly produced). The protocols known in the art may be used to isolate hsp-peptide complexes, or the hsps alone, from any eukaryotic cells for example, tissues, isolated cells, or immortalized eukaryote cell lines infected with a preselected intracellular pathogen, tumor cells or tumor cell lines.

Methods for (1) the preparation and purification of Hsp-70, Hsp-90, and gp96 peptide complexes; (2) isolation of antigenic/immunogenic. components of hsp-complexes and MHC-complexes; (3) isolation of peptides from stress protein-peptide and MHC-peptide complexes; (4) in vitro production of stress protein-antigenic molecule complexes; (5) methods for the rapid purification of peptide-associated hsp-70; and (6) determination of dose regimens incorporating methods for extrapolating appropriate human dosages from data obtained from animal models, are disclosed and described in detail in U.S. Pat. Nos. 5,832,251 and 5,935,576 issued to Srivastava, which are hereby incorporated by reference, in their entireties.

In various embodiments, the unfractionated cellular proteins used in combination with hsp-antigenic molecule complexes are unfractionated cytosolic soluble proteins.

### 4.3. Infectious Disease

In an alternative embodiment wherein it is desired to treat a patient having an infectious disease, unfractionated cytosolic soluble proteins are prepared from cells infected with an infectious agent, e.g., of a cell line or from a patient. Such infectious agents include but are not limited to, viruses, bacterial, protozoa, fungi, and parasites as described in detail hereinbelow. Furthermore, unfractionated cytosolic soluble proteins may be combined with hsp-peptide complexes that may either may be isolated from cells infected with the pathogenic agent of interest, or prepared in vitro by forming complexes of a heat shock protein and an antigenic peptide. The unfractionated cytosolic soluble proteins may also be prepared from recombinant cells expressing one or more protein or peptide antigens against which it is desired to generate an immune response. In various embodiments, the unfractionated cellular proteins used are unfractionated cytosolic soluble proteins.

### 4.3.1. Target Infectious Diseases

Infectious diseases that can be treated or prevented by the methods of the present invention are caused by infectious agents including, but not limited to, viruses, bacteria, fungi, protozoa and parasites.

Viral diseases that can be treated or prevented by the methods of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II).

Bacterial diseases that can be treated or prevented by the methods of the present invention are caused by bacteria including, but not limited to, mycobacteria rickettsia, mycoplasma, neisseria and legionella.

Protozoal diseases that can be treated or prevented by the methods of the present invention are caused by protozoa including, but not limited to, leishmania, kokzidioa, and trypanosoma.

Parasitic diseases that can be treated or prevented by the methods of the present invention are caused by parasites including, but not limited to, chlamydia and rickettsia.

### 4.4. Target Cancers

Cancers that can be treated or prevented by the methods of the present invention include, but not limited to human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenström's macroglobulinemia, and heavy chain disease. Specific examples of such cancers are described in the sections below.

In a specific embodiment the cancer is metastatic. In another specific embodiment, the patient having a cancer is immunosuppressed by reason of having undergone anti-cancer therapy (e.g., chemotherapy radiation) prior to administration of the compositions of the invention that comprise unfractionated cellular proteins.

### 4.4.1. Colorectal Cancer Metastatic to the Liver

It has been estimated that approximately 226,600 Americans will be diagnosed with cancers of the digestive tract in 2000. Most notably, the colon will be the primary site for approximately 93,800 of these cases and the rectum the primary site for another approximately 36,400 cases. Further, it is predicted that approximately 47,700 will die of colon cancer and another 8,600 will die of rectal cancer (Cancer Facts & Figures 2000, American Cancer Society (ACS), Atlanta, Ga., 2000) Eighty percent of patients who die of colon or rectal cancer have metastatic disease involving the liver. Most metastatic tumors of the liver are from gastrointestinal primaries. Unfortunately, the natural history of metastatic liver lesions carries a grave prognosis and systemic chemotherapy regimens have been unable to induce significant response rates or alter length of survival (Drebin, J. A., et al., in Current Therapy In Oncology, ed. J. E. Niederhuber, B. C. Decker, Mosby, 1993, p.426).

Colorectal cancer initially spreads to regional lymph nodes and then through the portal venous circulation to the liver, which represents the most common visceral site of metastasis. The symptoms that lead patients with colorectal cancer to seek medical care vary with the anatomical location of the lesion. For example, lesions in the ascending colon frequency ulcerate, which leads to chronic blood loss in the stool.

Radical resection offers the greatest potential for cure in patients with invasive colorectal cancer. Before surgery, the CEA titer is determined. Radiation therapy and chemotherapy are used in patients with advanced colorectal cancer. Results with chemotherapeutic agents (e.g., 5-fluorouracil) are mixed and fewer than percent of patients experience a greater than 50 percent reduction in tumor mass (Richards, 2d., F., et al., 1986, J. Clin. Oncol. 4:565).

Patients with widespread metastases have limited survival and systemic chemotherapy has little impact in this group of patients. In addition, systemically administered chemotherapy is often limited by the severity of toxicities associated with the various agents, such as severe diarrhea, mucositis and/or myelosuppression. Other techniques, including hepatic radiation, systemic chemotherapy, hepatic arterial ligation, tumor embolization and immunotherapy have all been explored, but, for the most part, have proven ineffectual in prolonging patient survival.

In a specific embodiment, the present invention provides compositions and methods for enhancing tumor specific immunity in individuals suffering from colorectal cancer metastasized to the liver, in order to inhibit the progression of the neoplastic disease. Preferred methods of treating these neoplastic diseases comprise administering an autologous composition comprising unfractionated cellular proteins, which elicits tumor-specific immunity against the tumor cells. Most specifically, the use of a composition of the invention, comprising unfractionated cellular proteins, could result in nearly complete inhibition of liver cancer growth in cancer patients, without inducing toxicity and thus providing a dramatic therapeutic effect.

Accordingly, as an example of the method of the invention, a composition comprising unfractionated cellular proteins, is administered to a patient diagnosed with colorectal cancer, with or without liver metastasis, via one of many different routes of administration, the preferred routes. being intradermal at different anatomical sites, e.g., left arm, right arm, left belly, right belly, left thigh, right thigh, etc. These routes of administration are used in sequence and the site of injection is varied for each weekly injection as described in Section 7. The preparations and use of therapeutically effective compositions for the prevention and treatment of primary and metastatic cancers are described in detail in the sections which follow and by way of example, infra.

### 4.4.2. Hepatocellular Carcinoma

Hepatocellular carcinoma is generally a disease of the elderly in the United States. Although many factors may lead to hepatocellular carcinoma, the disease is usually. limited to those persons with preexisting liver disease. Approximately 60 to 80 percent of patients in the United States with hepatocellular carcinoma have a cirrhotic liver and about four percent of individuals with a cirrhotic liver eventually develop hepatocellular carcinoma (Niederhuber, J. E., (ed.), 1993, Current Therapy in Oncology, B. C. Decker, Mosby). The risk is highest in patients whose liver disease is caused by inherited hemochromatosis or hepatic B viral infection (Bradbear, R. A., et al., 1985, J. Natl. Cancer Inst. 75:81; Beasley, R. P., et al., 1981, Lancet 2:1129). Other causes of cirrhosis that can lead to hepatocellular carcinoma include alcohol abuse and hepatic fibrosis caused by chronic administration of methotrexate. The most frequent symptoms of hepatocellular carcinoma are the development of a painful mass in the right upper quadrant or epigastrium, accompanied by weight loss. In patients with cirrhosis, the development of hepatocellular carcinoma is preceded by ascites, portal hypertension and relatively abrupt clinical deterioration. In most cases, abnormal values in standard liver function tests such as serum aminotransferase and alkaline phosphatase are observed.

CT scans of the liver are used to determine the anatomic distribution of hepatocellular carcinoma and also provide orientation for percutaneous needle biopsy. Approximately 70 percent of patients with hepatocellular carcinoma have an elevated serum alpha-fetoprotein concentration (McIntire, K. R., et al., 1975, Cancer Res. 35:991) and its concentration correlates with the extent of the disease.

Radical resection offers the only hope for cure in patients with hepatocellular carcinoma. Such operative procedures are associated with five-year survival rates of 12 to 30 percent. Liver transplantation may improve survival of some younger individuals. However, most patients are not surgical candidates because of extensive cirrhosis multifocal tumor pattern or scarcity of compatible donor organs.

Chemotherapeutic agents have been administered either by intravenous route or through an intrahepatic arterial catheter. Such therapy has sometimes been combined with irradiation to the liver. Reductions in the size of measurable tumors of 50% or more have been reported in some patients treated with either systemic doxorubicin or 5-fluorouracil. However, chemotherapy often induces immunosuppression and rarely causes the tumor to disappear completely and the duration of response is short. The prognosis for patients with hepatocellular carcinoma is negatively correlated with cirrhosis and metastases to the lungs or bone. Median survival for patients is only four to six months. In another specific embodiment, the present invention provides compositions and methods for enhancing specific immunity in individuals suffering from hepatocellular carcinoma in order to inhibit the progression of the neoplastic disease and ultimately irradiate all preneoplastic and neoplastic cells.

### 4.4.3. Breast Cancer

Another specific aspect of the invention relates to the treatment of breast cancer. The American Cancer Society estimated that in 2000, 184,200 American women will be diagnosed with breast cancer and 41,200 will succumb to the disease (Cancer Facts & Figures 2000, American Cancer Society (ACS), Atlanta, Ga., 2000). This makes breast cancer the second major cause of cancer death in women, ranking just behind lung cancer. The treatment of breast cancer presently involves surgery, radiation, hormonal therapy and/or chemotherapy. Consideration of two breast cancer characteristics, hormone receptors and disease extent, has governed how hormonal therapies and standard-dose chemotherapy are sequenced to improve survival and maintain or improve quality of life. A wide range of multidrug regimens have been used as adjuvant therapy in breast cancer patients, including, but not limited to combinations of 2 cyclophosphamide, doxorubicin, vincristine methotrexate, 5-fluorouracil and/or leucovorin. In a specific embodiment, the present invention provides compositions of unfractionated cellular proteins for enhancing specific immunity to preneoplastic and neoplastic mammary cells in women. The present invention also provides compositions of unfractionated cellular proteins and methods for preventing the development of neoplastic cells in women at enhanced risk for breast cancer, and for inhibiting cancer cell proliferation and metastasis. These compositions can be applied alone or in combination with each other or with biological response modifiers.

### 4.5. Autologous Embodiment

In a preferred embodiment of the invention directed to the use of autologous compositions comprising unfractionated cellular proteins, as cancer vaccines, two of the most intractable hurdles to cancer immunotherapy are circumvented. First is the possibility that human cancers, like cancers of experimental animals, are antigenically distinct. In an embodiment of the present invention, unfractionated cellular proteins or unfractionated cytosolic soluble proteins are derived from cancer cells and tissue of the patient to be treated, thereby circumventing this hurdle. Second, most current approaches to cancer immunotherapy focus on determining the CTL-recognized epitopes of cancer cell lines. This approach requires the availability of cell lines and CTLs against cancers. These reagents are unavailable for an overwhelming proportion of human cancers. In an embodiment of the present invention directed to autologous compositions comprising unfractionated cellular proteins, cancer immunotherapy does not depend on the availability of cell lines or CTLs nor does it require definition of the antigenic epitopes of cancer cells. These advantages make autologous compositions comprising unfractionated cellular proteins attractive and novel immunogens against cancer.

### 4.6. Prevention and Treatment of Primary and Metastatic Neoplastic Diseases

There are many reasons why immunotherapy as provided by the present invention is desired for use in cancer patients. First, where cancer patients are immunosuppressed, surgery, with anesthesia and subsequent chemotherapy, may worsen the immunosuppression, immunosuppression may be prevented or reversed by appropriate immunotherapy in the preoperative period. This could lead to fewer infectious complications and to accelerated wound healing. Second, tumor bulk is minimal following surgery and immunotherapy is most likely to be effective in this situation. A third reason is the possibility that tumor cells are shed into the circulation at surgery and effective immunotherapy applied at this time can eliminate these cells.

The preventive and therapeutic methods of the invention are directed at enhancing the immunocompetence of the cancer patient either before surgery, at or after surgery, and to induce tumor-specific immunity to cancer cells, with the objective being inhibition of cancer; and with the ultimate clinical objective being total cancer regression and eradication.

### 4.6.1. Monitoring of Effects During Cancer Prevention and Immunotherapy with Composition Comprising Unfractionated Cellular Proteins

The effect of immunotherapy with compositions comprising unfractionated cellular proteins, on development and progression of neoplastic diseases can be monitored by any method known to one skilled in the art, including but not limited to measuring: a) delayed hypersensitivity as an assessment of cellular immunity; b) activity of cytolytic T-lymphocytes in vitro; c) levels of tumor specific antigens, e.g., carcinoembryonic (CEA) antigens; d) changes in the morphology of tumors using techniques such as a computed tomographic (CT) scan; and e) changes in levels of putative biomarkers of risk for a particular cancer in individuals at high risk, and f) changes in the morphology of tumors using a sonogram.

The following subsections describe optional, exemplary procedures.

### 4.6.2. Delayed Hypersensitivity Skin Test

Delayed hypersensitivity skin tests are of great value in the overall immunocompetence and cellular immunity to an antigen. Inability to react to a battery of common skin antigens is termed anergy (Sato, T., et al., 1995, Clin. Immunol. Pathol. 74:35-43).

Proper technique of skin testing requires that the antigens be stored sterile at 4° C., protected from light and reconstituted shorted before use. A 25- or 27-gauge need ensures intradermal, rather than subcutaneous, administration of antigen. Twenty-four and 48 hours after intradermal administration of the antigen, the largest dimensions of both erythema and induration are measured with a ruler. Hypoactivity to any given antigen or group of antigens is confirmed by testing with higher concentrations of antigen or, in ambiguous circumstances, by a repeat test with an intermediate test.

### 4.6.3. Activity of Cytolytic T-lymphocytes In Vitro

8×10⁶ Peripheral blood derived T lymphocytes isolated by the Ficoll-Hypaque centrifugation gradient. technique, are restimulated with 4×10⁴ mitomycin C treated tumor cells in 3 ml RPMI medium containing 10% fetal calf serum. In some experiments, 33% secondary mixed lymphocyte culture supernatant or IL-2, is included in the culture medium as a source of T cell growth factors.

In order to measure the primary response of cytolytic T-lymphocytes after immunization, T cells are cultured without the stimulator tumor cells. In other experiments, T cells are restimulated with antigenically distinct cells. After six days, the cultures are tested for cytotoxicity in a 4 hour ⁵¹Cr-release assay. The spontaneous ⁵¹Cr-release of the targets should reach a level less than 20%. For the anti-MHC class I blocking activity, a tenfold concentrated supernatant of W6/32 hybridoma is added to the test at a final concentration of 12.5% (Heike M., et al., J. Immunotherapy 15:165-174).

### 4.6.4. Levels of Tumor Specific Antigens

Although it may not be possible to detect unique tumor antigens on all tumors, many tumors display antigens that distinguish them from normal cells. The monoclonal antibody reagents have permitted the isolation and biochemical characterization of the antigens and have been invaluable diagnostically for distinction of transformed from nontransformed cells and for definition of the cell lineage of transformed cells. The best-characterized human tumor-associated antigens are the oncofetal antigens. These antigens are expressed during embryogenesis, but are absent or very difficult to detect in normal adult tissue. The prototype antigen is carcinoembryonic antigen (CEA), a glycoprotein found on fetal gut an human colon cancer cells, but not on normal adult colon cells. Since CEA is shed from colon carcinoma cells and found in the serum, it was originally thought that the presence of this antigen in the serum could be used to screen patients for colon cancer. However, patients with other tumors, such as pancreatic and breast cancer, also have elevated serum levels of CEA. Therefore, monitoring the fall and rise of CEA levels in cancer patients undergoing therapy has proven useful for predicting tumor progression and responses to treatment.

Several other oncofetal antigens have been useful for diagnosing and monitoring human tumors, e.g., alpha-fetoprotein, an alpha-globulin normally secreted by fetal liver and yolk sac cells, is found in the serum of patients with liver and germinal cell tumors and can be used as a matter of disease status.

### 4.6.5. Computed Tomographic (CT) Scan

CT remains the choice of techniques for the accurate staging of cancers. CT has proved more sensitive and specific than any other imaging techniques for the detection of metastases.

### 4.6.6. Measurement of Putative Biomarkers

The levels of a putative biomarker for risk of a specific cancer are measured to monitor the effect of compositions comprising unfractionated cellular proteins. For example, in individuals at enhanced risk for prostate cancer, serum prostate-specific antigen (PSA) is measured by the procedure described by Brawer, M. K., et al., 1992, J. Urol. 147:841-845, and Catalona, W. J., et al., 1993, JAMA 270:948-958; or in individuals at risk for colorectal cancer CEA is measured as described above in Section 4.5.3; and in individuals at enhanced risk for breast cancer, 16-α-hydroxylation of estradiol is measured by the procedure described by Schneider, J. et al., 1982, Proc. Natl. Acad. Sci. ISA 79:3047-3051. The references cited above are incorporated by reference herein in their entirety.

### 4.6.7. Sonogram

A Sonogram remains an alternative choice of technique for the accurate staging of cancers.

### 5. EXAMPLE

### Characterization of Composition Comprising Unfractionated Cellular Proteins

5.1. Measuring Generation of MHC Class I Restricted CD8⁺ CTLs Provides An Assay For In Vivo Tumor Rejection

The effect of vaccination with compositions comprising unfractionated cellular proteins, can be measured by tumor rejection assays in vivo. While this assay is clearly the most demanding and rigorous evidence for immunogenicity, it is impractical for the purpose of monitoring immune response in humans. The ability of tumor-derived compositions comprising unfractionated cellular proteins, to elicit a CD8⁺ T cell response is evaluated in order to define an in vitro correlate for in vivo tumor rejection.

Mice are immunized twice with a composition comprising 10² to 10⁶ cell equivalents of unfractionated cellular proteins derived from 6138 or 6139SJ cells (Ward et al., 1989, J. Exp. Med. 170:217). Mixed lymphocyte-tumor cultures (MLTCs) are generated from immunized mice and tested in a ⁵¹Chromium release assay to assay for tumor-specific cytotoxicity for the tumor used as the source of the composition comprising unfractionated cellular proteins. This cytotoxic activity is blocked by anti-MHC class I antibody K44 (Ozato, K., et al., 1985, Proc. Natl. Acad. Sci. USA 82:2427) and by anti-CD8 antibody YTS169.4 (Cobbold, S. P., et al., 1984, Nature 312:548). No corresponding activity is expected in MLTCs generated from spleens of naive mice. Such results demonstrate that vaccination with a composition comprising 10² to 10⁶ cell equivalents of unfractionated cellular proteins, elicits an effective tumor-specific CTL response, which may be measured in vitro.

While testing the ability of a tumor-derived unfractionated cellular protein preparations to elicit CTL responses, vaccination with irradiated whole tumor cells is carried out as a positive control. Vaccination with intact irradiated 6138 cells normally leads to a vigorous tumor-specific CTL response.

### 5.2. Analysis of the Ability of Compositions Comprising Unfractionated Cellular Proteins to Elicit a Memory T Cell Response

The ability to elicit a memory response is crucial for any vaccine and, therefore, the ability of a composition comprising unfractionated cellular proteins to elicit a memory T cell population is tested. A number of criteria, i.e., radiation resistance, kinetics of appearance, loss of CD45RB and L-selectin lymphocyte surface antigens, are used to identify memory T response. In contrast to naive T cells (Schrek, R., 1961, Ann. N.Y. Acad. Sci 95:839), memory T cells are cycling cells (Mackay, C. R., et al., 1992, Nature 360:264) and like other cycling lymphocytes, are resistant to sub-lethal irradiation (Lowenthal, J. W., et al., 1991, Leuc. Biol. 49:388). Thus, radiation-resistance is used to distinguish naive resting T cells from activated effector and memory T cells. However, no known surface markers distinguish activated effector T cells from memory T cells and the two are distinguishable only by the kinetics of their appearance. Activated effector T cells disappear from circulation within seven to ten days of depletion of significant quantities of antigen (Sprent, J., 1994, Cell 76:315); in contrast, memory T cells continue to circulate well beyond this window of time. In order to test if vaccination with tumor-derived unfractionated cellular proteins elicits a memory T cell response, mice are vaccinated twice at ten day intervals, with a composition comprising unfractionated cellular proteins and the vaccinated mice are irradiated (400 rad) twelve days after the last vaccination. Three days after irradiation, MLTCs are generated from spleens of mice and tested for tumor-specific CTL response. It is expected that the irradiated, mice vaccinated with unfractionated cellular proteins generate powerful, MHC class I—restricted and tumor-specific CTL responses. Under this regimen of vaccination and irradiation, the irradiation eliminates the non-memory resting T cells, while the delay between the last vaccination and generation of MCTCs eliminates activated T lymphocytes (Sprent, J., 1994, Cell 76:315). Therefore, under these conditions, detection of a desired CTL response indicates that the composition comprising unfractionated cellular proteins elicits a response derived from radiation-resistant memory T cells. This phenomenon is also tested in tumor rejection assays in vivo. Mice vaccinated with a composition comprising unfractionated cellular proteins are irradiated and then observed to evaluate their resistance to tumor challenges for up to 17 days after vaccination. Tumor rejection under these conditions indicates that vaccination with a composition comprising unfractionated cellular proteins elicits a long-lived, radiation-resistant T cell population.

As an independent parameter for memory response, expression of CD45RB (Birkeland, M. L., et al., 1989, Proc. Natl. Acad. Sci. USA 86:6734) on CD8⁺ lymphocytes from irradiated and non-irradiated, naive mice and mice vaccinated with a composition comprising unfractionated cellular proteins are also tested. In each case, lymphocytes are obtained under the same regimen as described in the preceding paragraph, i.e., fifteen days after the last vaccination including three days after irradiation, in order to allow the activated effector cells to be depleted. If the results indicate that vaccination with a composition comprising unfractionated cellular proteins leads to relative loss of expression of CD45RB on CD8⁺ T lymphocytes in irradiated as well as non-irradiated, immunized mice, it suggests that this vaccination elicits a memory T cell response. These results are similar to those observed with L-selectin. Such results indicate that as judged from two independent sets of criteria, vaccination with a composition comprising unfractionated cellular proteins elicits a memory T cell response.

### 6. EXAMPLE

### Administration of a Composition Comprising Unfractionated Cellular Proteins in the Treatment of Hepatocellular Carcinoma

Patients with hepatocellular carcinoma are injected with a composition comprising unfractionated cellular proteins (derived from their own tumors or from other tumors) post surgery. Treatment with a composition comprising unfractionated cellular proteins is started any time after surgery. However, if the patient has received chemotherapy, compositions comprising unfractionated cellular proteins are usually administered after an interval of four weeks or more so as to allow the immune system to recover. The immunocompetence of the patient is tested by procedures well known in the art or as described herein.

The therapeutic regimen of compositions comprising unfractionated cellular proteins, includes weekly injections of the composition comprising unfractionated cellular proteins dissolved in saline or other physiologically compatible solution.

The dosage used is in the range of from about 10² to about 10⁹ cell equivalents, preferably in the range of from about 10² to about 5×10⁷ cell equivalents, and more preferably in the range of from about 10³ to about 5×10⁵ cell equivalents of cell unfractionated cellular proteins.

The route and site of injection are varied each time, for example, the first injection is given subcutaneously on the left arm, the second injection on the right arm, the third injection on the left abdominal region, the fourth injection on the right abdominal region, the fifth injection on the left thigh, the sixth injection on the right thigh, etc. The same site is repeated after a gap of one or more injections. In addition, injections are split and each half of the dose is administered at a different site on the same day.

Overall, the first four to six injections are given at weekly intervals. Subsequently, two injections are given at two-week intervals; followed by a regimen of injections at monthly intervals. The effect of treatment with a composition comprising unfractionated cellular proteins is monitored by measuring: a) delayed hypersensitivity as an assessment of cellular immunity; b) activity of cytolytic T-lymphocytes in vitro; c) levels of tumor specific antigens, e.g., carcinoembryonic (CEA) antigens; d) changes in the morphology of tumors using techniques such as a computed tomographic (CT) scan; and e) changes in putative biomarkers of risk for a particular cancer in individuals at high risk.

Depending on the results obtained, as described above Section 4.6, the therapeutic regimen is developed to maintain and/or boost the immunological responses of the patient, with the ultimate goal of achieving tumor regression and complete eradication of cancer cells.

### 7. EXAMPLE

### Administration of Compositions Comprising Unfractionated Cellular Proteins in the Treatment of Colorectal Cancer

Compositions comprising unfractionated cellular proteins are administered as adjuvant therapy and as prophylactic adjuvant therapy in patients after complete reduction of colorectal cancer to eliminate undetectable micrometastases and to improve survival.

The therapeutic and prophylactic regimens used in patients suffering from colorectal cancer are the same as those described in Section 6 above for patients recovering with hepatocellular carcinoma. The methods of monitoring of patients under clinical evaluation for prevention and treatment of colorectal cancer is done by procedures described in Section 4.6.4. Specifically, CEA levels are measured as a useful monitor of tumor regression and/or recurrence (Mayer, R. J., et al., 1978, Cancer 42:1428).

### 8. EXAMPLE

### Immunotherapy of Meth a Tumors by Administration of Unfractionated Cellular Proteins

Tumor Model:

The Meth A tumor model is a methylcholanthrene-induced tumor that was originally isolated from a BALB/c mouse, and is now maintained by serial passage of ascites fluid in BALB/c mice (Old et al. Ann. N.Y. Acad. Sci. 101: 80-67 (1962)).

Preparation of Unfractionated Cellular Proteins:

Compositions comprising unfractionated cellular proteins, were prepared from Meth A tumors according to the procedures described above in Section 4.1.1. The compositions comprising unfractionated cellular proteins, preparations are administered without adjuvants. In this example, clarified Meth A tumor cell extracts were prepared by freezing and thawing cell suspensions that were then subjected centrifugation at only low speed, i.e., 1000×g, to provide a supernatant comprising unfractionated cellular proteins, which were then administered to tumor-bearing mice for treatment.

Treatment

Materials and Method:

The ability of compositions comprising unfractionated cellular proteins derived from Meth A tumor cells to induce regression Meth A tumors in vivo was tested. A total of seven groups, with each group consisted of ten female BALB/c mice (obtained from The Jackson Laboratory, Bar Harbor, Me.), weighing approximately 25 g each, were used.

In each set, mice were injected intradermally with 10⁵ Meth A cells. Beginning five days after injection of the tumor cells (day 5), each group of mice was administered PBS buffer, irradiated whole Meth A tumor cells, 1×10³, 1×10⁴, 1×10⁵, 1×10⁶, or 1×10⁷ cell equivalents of unfractionated cellular proteins prepared from Meth A tumor cells. These treatments were repeated for each group of mice at day 7, 9, 12, 14, and 16.

Results

Average tumor diameter (in mm) was determined daily for each mouse until day 25, and the results are presented in Table 1. The data below indicate that unfractionated cellular proteins isolated from a tumor treatment can be used for treatment of that tumor in vivo.

TABLE I
Immunotherapy of Mice Injected with Meth A Tumor
Cells Using Unfractionated Cellular Proteins

| | Number of Mice Without Tumor/ |
|---|---|
| Composition Administered | Total Number of Mice Tested |
| PBS Buffer | 1/10 |
| Irradiated Meth A Tumor Cells | 0/10 |
| Unfractionated Cellular Proteins: | |
| 1 × 10³ cell equivalents | 2/10 |
| 1 × 10⁴ cell equivalents | 0/10 |
| 1 × 10⁵ cell equivalents | 1/10 |
| 1 × 10⁶ cell equivalents | 4/10 |
| 1 × 10⁷ cell equivalents | 3/10 |

Prevention

Materials and Method:

The ability of compositions comprising unfractionated cellular proteins derived from Meth A tumor cells to prevent development of Meth A tumors in vivo was tested. A total of thirteen groups, with each group consisting of five female BALB/c mice (obtained from The Jackson Laboratory, Bar Harbor, Me.), weighing approximately 25 g each, were used.

In each set, mice were vaccinated intradermally at day 0 and day 7 with the indicated amount (in cell equivalents) of unfractionated cellular proteins, which were prepared by lysing Meth A tumor cells by freezing and thawing, Dounce homogenization, and sonication as indicated, and subjecting the lysate to centrifugation only at low-speed (1,000×g). On day 14, mice were challenged by intradermal injection of 10⁵ viable Meth A tumor cells.

Results

The presence of palpable tumors was evaluated every three to four days until day 25, and the number of mice that had rejected the tumor was determined. The data obtained are summarized below in Table II. These data indicate that unfractionated cellular proteins isolated from a tumor, using methods comprising only low-speed centrifugation, can be used for vaccination against that tumor in vivo.

TABLE II
Tumor Rejection in Mice Vaccinated With
Unfractionated Cellular Proteins

| | Number of Mice Without Tumor/ |
|---|---|
| Composition Administered | Total Number of Mice Tested |
| PBS Buffer | 1/5 |
| Unfractionated Cellular Protein Prepared Using Only Low-speed | |
| Centrifugation; Cells Lysed by Freezing and Thawing: | |
| 1 × 10⁵ cell equivalents | 1/5 |
| 1 × 10⁶ cell equivalents | 5/5 |
| 1 × 10⁷ cell equivalents | 4/5 |
| Unfractionated Cellular Protein Prepared Using Only Low-speed | |
| Centrifugation, Cells Lysed by Dounce Homogenization: | |
| 1 × 10⁴ cell equivalents | 0/5 |
| 1 × 10⁵ cell equivalents | 5/5 |
| 1 × 10⁶ cell equivalents | 3/5 |
| 1 × 10⁷ cell equivalents | 5/5 |
| Unfractionated Cellular Protein Prepared Using Only Low-speed | |
| Centrifugation; Cells Lysed by Sonication: | |
| 1 × 10³ cell equivalents | 1/5 |
| 1 × 10⁴ cell equivalents | 0/5 |
| 1 × 10⁵ cell equivalents | 3/5 |
| 1 × 10⁶ cell equivalents | 4/5 |
| 1 × 10⁷ cell equivalents | 4/5 |

The present invention is not to be limited in scope the specific embodiments described herein. Indeed, various modifications of the invention in addition to those described herein will become apparent to those skilled in the art from the foregoing description. Such modifications are intended to fall within the scope of the appended claims.

Various references are cited herein, including patents, patent applications, and scientific literature, the disclosures of which are incorporated by reference in their entireties for all purposes.

What is claimed is:

1. A method of inducing an immune response in a subject against a type of cancer, comprising administering to said subject a composition comprising an immunogenic amount of unfractionated cellular proteins obtained from cells of said type of cancer or a metastasis thereof effective to induce said immune response.

2. The method of claim 1 wherein the unfractionated cellular proteins are from 10⁷ cell equivalents or less of said cells.

3. The method of claim 1 wherein the unfractionated cellular proteins are from 10⁶ cell equivalents or less of said cells.

4. A method of treating or preventing a type of cancer, comprising administering to a subject in need of such treatment or prevention a composition comprising an amount, effective for said treatment or prevention, of unfractionated cellular proteins obtained from cells of said type of cancer or a metastasis thereof.

5. The method of claim 4 wherein the unfractionated cellular proteins are from 10⁷ cell equivalents or less of said cells.

6. The method of claim 4 wherein the unfractionated cellular proteins are from 10⁶ cell equivalents or less of said cells.

7. The method of claim 1 or 4, wherein the unfractionated cellular proteins are contained in a solution substantially free of cell membrane.

8. The method of claim 1 or 4, wherein the unfractionated cellular proteins are contained in a solution substantially free of cell organelles or particles thereof.

9. The method of claim 1 or 4, wherein the unfractionated cellular proteins are contained in a solution substantially free of viral particles.

10. The method of claim 1 or 4, wherein the unfractionated cellular proteins are from 5×10⁵ cell equivalents or less of said cells.

11. The method of claim 10, wherein the unfractionated cellular proteins are from 10⁴ cell equivalents or less of said cells.

12. The method of claim 11, wherein the unfractionated cellular proteins are from 10³ cell equivalents or less of said cells.

13. The method of claim 10, wherein the unfractionated cellular proteins are from 10² to 5×10⁵ cell equivalents of said cells.

14. The method of claim 1 or 4, wherein the unfractionated cellular proteins are prepared by a method comprising subjecting a lysed sample of said cells to centrifugation one or more times with the highest force being 1,000×g, and substantially not subjecting proteins within the lysed sample to any method that selectively removes proteins.

15. The method of claim 1 or 4, wherein the unfractionated cellular proteins are unfractionated cytosolic soluble proteins prepared by a method comprising subjecting a lysed sample of said cells to centrifugation one or more times with the highest force being about 100,000×g, and substantially not subjecting cytosolic, soluble proteins within the lysed sample to any method that selectively removes soluble proteins.

16. The method of claim 1 or 4, wherein the cells from which the proteins are obtained are autologous to the subject.

17. The method of claim 1 or 4, wherein the cells from which the proteins are obtained are allogeneic to the subject.

18. The method of claim 1 or 4, wherein said cells are obtained from a tumor.

19. The method of claim 1 or 4, wherein said cells are of a tumor cell line.

20. The method of claim 1 or 4, wherein the composition further comprises an adjuvant.

21. The method of claim 1 or 4, wherein the composition is substantially free of adjuvant.

22. The method of claim 1 or 4, wherein said administering is repeated at weekly intervals.

23. The method of claim 1 or 4, wherein said administering is repeated at the same site of the subject.

24. The method of claim 1 or 4, wherein said administering is repeated at different sites.

25. The method of claim 1 or 4, wherein the composition is administered intradermally.

26. The method of claim 1 or 4, wherein the composition is administered subcutaneously.

27. The method of claim 1 or 4, wherein the type of cancer is a sarcoma or carcinoma, selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenström's macroglobulinemia, and heavy chain disease.

28. A method of inducing an immune response in a subject against a pathogen, comprising administering to said subject a composition comprising an immunogenic amount of unfractionated cellular proteins obtained from cells having an antigenicity of said pathogen.

29. A method of treating or preventing an infection by a pathogen in a subject comprising administering to a subject in need of such treatment or prevention a composition comprising an amount, effective for such treatment or prevention, of unfractionated cellular proteins obtained from cells having an antigenicity of said pathogen.

30. The method of claim 28 or 29 wherein the proteins are obtained from cells infected with an agent displaying the antigenicity of said pathogen or from cells transformed with and expressing a nucleic acid displaying the antigenicity of said pathogen.

31. The method of claim 28 or 29 wherein the unfractionated cellular proteins are from 10⁷ cell equivalents or less of said cells.

32. The method of claim 28 or 29 wherein the unfractionated cellular proteins are from 10⁶ cell equivalents or less of said cells.

33. The method of claim 1, 2, 3, 4, 28 or 29, wherein the subject is human.

34. The method of claim 28 or 29, wherein the unfractionated cellular proteins are contained in a solution substantially free of cell membrane.

35. The method of claim 28 or 29, wherein the unfractionated cellular proteins are contained in a solution substantially free of cell organelles or particles thereof.

36. The method of claim 28 or 29, wherein the unfractionated cellular proteins are from 5×10⁵ cell equivalents or less of said cells.

37. The method of claim 36, wherein the unfractionated cellular proteins are from 10⁴ cell equivalents or less of said cells.

38. The method of claim 37, wherein the unfractionated cellular proteins are from 10³ cell equivalents or less of said cells.

39. The method of claim 36, wherein the unfractionated cellular proteins are from 10² to 5×10⁵ cell equivalents of said cells.

40. The method of claim 28 or 29, wherein the unfractionated cellular proteins are prepared by a method comprising subjecting a lysed sample of said cells to centrifugation one or more times with the highest force being 1,000×g, and substantially not subjecting proteins within the lysed sample to any method that selectively removes proteins.

41. The method of claim 28 or 29, wherein the unfractionated cellular proteins are unfractionated cytosolic soluble proteins prepared by a method comprising subjecting a lysed sample of said cells to centrifugation one or more times with the highest force being about 100,000×g, and substantially not subjecting cytosolic, soluble proteins within the lysed sample to any method that selectively removes soluble proteins.

42. The method of claim 28 or 29, wherein the cells from which the proteins are obtained are autologous to the subject.

43. The method of claim 28 or 29, wherein the cells from which the proteins are obtained are allogeneic to the subject.

44. The method of claim 28 or 29, wherein the composition further comprises an adjuvant.

45. The method of claim 28 or 29, wherein the composition is substantially free of adjuvant.

46. The method of claim 28 or 29, wherein said administering is repeated at weekly intervals.

47. The method of claim 28 or 29, wherein said administering is repeated at the same site of the subject.

48. The method of claim 28 or 29, wherein said administering is repeated at different sites of the subject.

49. The method of claim 28 or 29, wherein the composition is administered intradermally.

50. The method of claim 28 or 29, wherein the composition is administered subcutaneously.

51. The method of claim 28 or 29, wherein the pathogen is selected from the group consisting of hepatitis virus type A, hepatitis virus type B, hepatitis virus type C, influenza virus, varicella virus, adenovirus, herpes simplex virus type I (HSV-I), herpes simplex virus type II (HSV-II), rinderpest virus, rhinovirus, echovirus, rotavirus, respiratory syncytial virus, papilloma virus, papova virus, cytomegalovirus, echinovirus, arbovirus, hantavirus, coxsackie virus, mumps virus, measles virus, rubella virus, polio virus, human immunodeficiency virus type I (HIV-I), human immunodeficiency virus type II (HIV-II), mycobacteria rickettsia, mycoplasma, neisseria, legionella, leishmania, kokzidioa, trypanosoma, chlamydia and rickettsia.

52. The method of claim 28 or 29, wherein the composition further comprises at least one biological response modifier selected from the group consisting of heat shock proteins, interferons, interleukins, colony stimulating factors, monoclonal antibodies, and tumor necrosis factor.

53. The method of claim 29, wherein said proteins are obtained from infected cells.

54. The method of claim 28 or 29, wherein the pathogen is a virus.

55. The method of claim 28 or 29, wherein the pathogen is a bacterium.

56. The method of claim 28 or 29, wherein the pathogen is a parasite.

57. The method of claim 4, which is for treating a type of cancer.

58. The method of claim 29, which is for treating an infection by a pathogen.

59. The method of claim 57 or 58, wherein the subject is a human in need of such treatment.

60. A method for preparing a vaccine for treatment or prevention of cancer comprising:
(a) lysing cancer cells to produce a crude cell lysate;
(b) centrifuging said crude cell lysate or supernatant derived therefrom one or more times to remove intact cells;
wherein there is substantially no subjecting of cellular proteins within said lysate to any method that selectively removes soluble proteins.

61. The method of claim 60, wherein said lysing step is done by using hypotonic shock combined with mechanical disruption.

62. The method of claim 60, wherein said lysing step comprises disruption by freezing and thawing the cancer cells.

63. The method of claim 60 or 61, wherein said centrifuging step comprises centrifuging at 1,000×g to produce a supernatant.

64. The method of claim 60 or 61, wherein said centrifuging step comprises a first centrifuging at 1,000×g to produce a first supernatant, and a second centrifuging at 100,000×g of said first supernatant to produce a second supernatant, wherein said second supernatant is substantially free of cell membranes and organelles.

65. The method of claim 64 which further comprises dialyzing said second supernatant against a suitable buffer.

66. A method of treating or preventing a type of cancer, comprising administering to a subject in need of such treatment or prevention a composition comprising an amount, effective for said treatment or prevention, of unfractionated cytosolic soluble proteins obtained from cells transformed with and expressing a nucleic acid encoding a molecule displaying antigenicity of a tumor-associated antigen or tumor-specific antigen of said type of cancer.

67. A kit comprising in one or more containers an amount, effective for treatment or prevention of a type of cancer, of unfractionated cellular proteins obtained from cells of said type of cancer or a metastasis thereof or from cells transformed with and expressing a nucleic acid encoding a molecule displaying antigenicity of a tumor-associated antigen or tumor-specific antigen of said type of cancer.

68. A kit comprising in one or more containers an amount, effective for treatment or prevention of an infectious disease, of unfractionated cellular proteins obtained from cells having an antigenicity of a pathogen that causes the infectious disease.

69. The kit of claim 68 wherein the proteins are obtained from cells infected with an agent displaying the antigenicity of said pathogen or from cells transformed with and expressing a nucleic acid displaying the antigenicity of said pathogen.
